(19) United States
(12) Patent Application Publication
(10) Pub. No.: US 2024/0207248 A1
(43) Pub. Date: Jun. 27, 2024

(54) METHODS, KITS AND COMPOSITIONS FOR REDUCING CHROMOSOMAL INSTABILITY IN CANCER CELLS

(71) Applicant: BARUCH S. BLUMBERG INSTITUTE, Doylestown, PA (US)

(72) Inventors: Richard G. PESTELL, Fort Lauderdale, FL (US); Yanming DU, Cheshire, CT (US)

(21) Appl. No.: 18/287,988

(22) PCT Filed: Apr. 25, 2022
(86) PCT No.: PCT/US2022/026221

§ 371 Date: Oct. 23, 2023

Related U.S. Application Data
(60) Provisional application No. 63/178,633, filed on Apr. 23, 2021.

(51) Int. Cl. A61K 31/4741; A61K 31/352; A61K 45/06; A61P 43/00

(52) U.S. Cl. CPC A61K 31/4741; A61K 31/352; A61K 45/06; A61P 43/00

(57) ABSTRACT

Described herein are methods for administering a chemotherapeutic agent to a patient in need thereof. Such methods may treat, prevent, or ameliorate tumor CIN associated with administration of a chemotherapeutic agent, resulting in therapeutic resistance, tumor progression and death also described are kits and compositions useful to implement the methods.

### CROSS-REFERENCE TO RELATED APPLICATION

This application claims the benefit of priority from U.S. Provisional Application No. 63/178,633, filed Apr. 23, 2021, the contents of which are hereby incorporated herein by reference in their entirety

### STATEMENT REGARDING GOVERNMENT INTERESTS

This invention was made with government support under R01-CA137494-05 awarded by the National Institutes of Health and Breast Cancer Research, Breakthrough (Pestell PI) No. W81XWH1810605. The government has certain rights in the invention.

### BACKGROUND

Chromosomal instability in tumorigenesis. The enhanced survival of cancer patients (>30% survival 5 years beyond initial diagnosis), is due in part to the use of chemotherapy and radiation. Unfortunately, chemotherapy and radiation are often associated with increased chromosomal instability.

CIN as a therapeutic target. Aneuploidy enhances sensitivity to compounds that induce phototoxic and energy stress. The NCI-60 drug discovery panel of human cancer cell lines was analyzed to uncover potential anticancer agents that specifically target cancer cells with higher levels of chromosomal instability (“CIN”). Although seven groups of compounds were found that could preferentially kill high CIN cell lines, these compounds are also known to increase CIN. A recent study identified three energy and proteotoxic stress-inducing compounds—AICAR, 17-AAG and chloroquine showed selectivity against aneuploid primary mouse embryonic fibroblasts carrying Robertsonian fusion chromosomes.

Tumor proliferation and growth has been used as an important clinical end point for cancer treatment. Tumor recurrence however ultimately determines survival. Tumor recurrence and therapy resistance is driven in part by tumor heterogeneity, which is driven in part by genomic and chromosomal instability (“CIN”) and the presence of cancer stem cells (“CSC”). Reducing CIN is important as CIN can drive intra-tumor heterogeneity, multi drug resistance and cancer stem cells and CIN is correlated with poor prognosis in multiple clinical studies of breast cancer. cyclin D1 induced chromosomal instability CIN, and CIN is widely considered to be a protumorigenic activity in breast cancer. Cyclin D1 induced CIN in the presence of Cdk inhibitors. CIN may be sufficient for the induction of tumorigenesis and is important in therapeutic responses to specific therapies.

Most cancer therapeutics reduce proliferation, but in recent studies of more than 62 anticancer drugs, used at the IC₅₀ that reduced proliferation, the same agents induced CIN. In our recent studies, we showed that cyclin D1, which is overexpressed in more than 50% of human breast cancer, induces both cellular proliferation and chromosomal instability (FIG. 1B). Conversely a reduction in cyclin D1 reduced both proliferation and CIN. Cyclin D1 antisense reduced proliferation and CIN in the mammary gland of mice in vivo.

Data herein show cyclin D1 promotes chromosomal instability and expression of high CIN score genes through direct association with chromatin. We have identified a conserved C-terminal domain in cyclin D1 that associates primarily with histone H3, deletion of the domain abrogates cyclin D1 binding to promoter regulatory elements of genes. From our structural and surface analysis of the crystal structure of CDK4-cyclin-D complex we have identified a drugable pocket. Our hypothesis is that a small molecule bound to the identified pocket in cyclin-D1 will change the position of the negatively charged C-terminal domain such that it will disrupt the binding of cyclin-D1 to chromatin. In-silico screening of small organic compounds from the publicly available databases PubChem based on interaction energy and surface-based scoring function identified a compound termed JDB-1782.

Accordingly, there remains a critical need for methods, kits and compositions that are able to effectively reduced CIN in cancer cells including CIN associated with current therapies given for cancer. Embodiments of the present invention are designed to meet these and other needs.

### SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In some embodiments, the present invention provides a method for administering a compound (CIN antagonist) to a patient receiving a chemotherapeutic agent who is in need thereof comprising administering an effective amount of a CIN antagonist followed by administering an effective amount of a chemotherapeutic agent.

In other embodiments, the present invention provides a method of treating, preventing, or ameliorating a symptom associated with, the CIN resulting from the administration of a chemotherapeutic agent comprising administering an effective amount of a CIN antagonist followed by administering an effective amount of a chemotherapeutic agent.

Still further embodiments of the present invention provide a method of enhancing chemotherapy function in a patient in need thereof comprising administering an effective amount of a CIN antagonist followed by administering an effective amount of a chemotherapeutic agent.

While other embodiments of the present invention provide a method of increasing survival rate or extending survival time in a patient undergoing treatment with a chemotherapeutic agent comprising administering an effective amount of a CIN antagonist followed by administering an effective amount of a chemotherapeutic agent.

Some embodiments of the present invention provide a method for reducing the effective dose of a chemotherapeutic agent in a patient in need thereof comprising administering an effective amount of a CIN antagonist followed by administering an effective amount of a chemotherapeutic agent.

Certain embodiments of the present invention provide a method for reducing the CIN associated with a chemotherapy, comprising co-administering an effective amount of a CIN antagonist and a chemotherapeutic agent. In some embodiments, the CIN antagonist and chemotherapeutic agent are administered contemporaneously. In some embodiments, the CIN antagonist is administered prior to the chemotherapeutic agent. In some embodiments, the CIN antagonist is administered from about 1 minute to about 72 hours prior to administration of the chemotherapeutic agent, optionally about 15 minutes, or 30 minutes, or 60 minutes, 90 minutes, or 2 hours, or 4 hours, or 8 hours, or 12 hours, or 18 hours, or 24 hours, or 36 hours, or 48 hours, or 60 hours or 72 hours, prior to administration of the chemotherapeutic agent. In some embodiments, the method further comprises the step of administering an additional dose of a CIN antagonist following administration of the chemotherapeutic agent.

Further areas of applicability of the present invention will become apparent from the detailed description provided hereinafter. It should be understood that the detailed description and specific examples, while indicating the typical embodiments of the invention, are intended for purposes of illustration only and are not intended to limit the scope of the invention.

### BRIEF DESCRIPTION OF THE DRAWINGS

The detailed description of the invention will be better understood when read in conjunction with the appended drawings. It should be understood, however, that the invention is not limited to the precise arrangements and instrumentalities of the embodiments shown in the drawings.

FIG. 1A depicts a Top2A mRNA abundance in the mammary gland of Tet-inducible cyclin D1 mammary epithelial cell targeted transgenic mice (7 days of transgenic induction) or MMTV-cyclin D1 transgenic induced mammary gland tumors according to aspects of the invention;

FIG. 1B depicts a Western blot for Top2a and B tubulin abundance in cyclin D1^{-/-} 3T3 cells rescued with a cyclin D1 expression vector with quantitation of multiplicate experiments in accordance with aspects of the invention;

FIG. 1C depicts a Top2a intensity assessed by immunofluorescent staining in cyclin D1^{-/-} cyclin D1 rescue 3T3 cells according to aspects of the invention;

FIG. 1D depicts an endogenous murine LPL ChIP assay in accordance with aspects of the invention;

FIG. 1E depicts Top2A ChIP assays with antibodies directed to target proteins according to aspects of the invention;

FIGS. 1F-1L are bar graphs of real-time quantitative PCR (qPCR) for the percent of input of Top2A in accordance with aspects of the invention;

FIG. 2A is a heat map depiction of samples from combined breast cancer microarray datasets that were assigned to the five breast cancer microarray subtypes according to aspects of the invention;

FIG. 2B depicts relative enrichment of TOP2A in ERα⁺ vs. ERα⁻ breast cancer in accordance with aspects of the invention;

FIGS. 2C-2G are scatter plots depict CCND1 transcript level versus TOP2A, showing the relationship between TOP2A and cyclin D1 expression in luminal B and normal-like subtype-specific according to aspects of the invention;

FIG. 3A is a schematic representation of cyclin D1^{wt} and cyclin D1^{ΔE} mutant in accordance with aspects of the invention;

FIG. 3B depicts a Western blot detection of the FLAG-tagged expression vectors according to aspects of the invention;

FIG. 3C depicts densitometric analysis of cyclin D1 proteins in accordance with aspects of the invention;

FIG. 3D depicts immunofluorescence staining of the FLAG epitope according to aspects of the invention;

FIG. 3E depicts Mammalian 2-hybrid interaction of cyclin D1 wt and cyclin D1^{ΔE} in accordance with aspects of the invention;

FIG. 3F depicts ChIP assays of the FLAG-tagged cyclin D1 proteins according to aspects of the invention;

FIG. 3G is a bar graph depicting occupancy of FLAG-tagged cyclin D1 proteins in accordance with aspects of the invention;

FIG. 3H depicts a schematic representation of cyclin D1^{wt} and cyclin D1^{ΔE} with representative ChIP analysis of the cyclin D1 binding site according to aspects of the invention;

FIG. 4A depicts a schematic representation of cyclin D1^{wt} and mutants in accordance with aspects of the invention;

FIG. 4B depicts Karyotype determined through SKY analysis for cyclin D1^{-/-} cells rescued with either cyclin D1^{wt} or cyclin D1 mutants according to aspects of the invention;

FIG. 4Cis a bar graph of the mean polyploidy associated with the cyclin D1^{-/-} cells rescued with either cyclin D1^{wt} or cyclin D1 mutants of FIG. 4B;

FIG. 4D depicts Top2A promoter luciferase reporter assays in accordance with aspects of the invention;

FIG. 4E is a graph showing cellular proliferation assays conducted in cyclin D1^{-/-} 3T3 cells, rescued either with cyclin D1^{wt}, mutant or Control vector according to aspects of the invention;

FIG. 4F depicts a summary of cyclin D1 mutant properties in accordance with aspects of the invention;

FIG. 5A depicts a disorder tendency and disordered binding site prediction according to aspects of the invention;

FIG. 5B depicts a multiple sequence alignment of the last (carboxyl-terminal) 100 amino acids of cyclin D1 from various species and of human cyclin A1, B1 and E1 in accordance with aspects of the invention;

FIG. 5C depicts an E-box motif found in 149 human proteins by BLASTP, and their enrichment in GO Biological Process (BP) according to aspects of the invention;

FIG. 6A depicts a Coomassie staining for cyclin D1 (CD1), cyclin D1 E-box deleted (CD1 ΔE) and KDM4A GST fusion proteins in accordance with aspects of the invention;

FIG. 6B depicts a Western blot for cyclin D1 (CD1), cyclin D1 E-box deleted (CD1 ΔE) and KDM4A GST fusion proteins according to aspects of the invention;

FIG. 6C depicts a GST pulldown of histone peptide (H2B) and S14-phosphorylated histone peptide (H2BS14P) in accordance with aspects of the invention;

FIG. 6D is a bar graph of the binding rate for GST-CD1, GST-CD1ΔE or GST-ctrl fusion proteins with histone peptide (H2B) and S14-phosphorylated histone peptide (H2BS14P) according to aspects of the invention;

FIG. 6E depicts a GST pull-down of histone H3K4me3 with KDM4A in accordance with aspects of the invention;

FIG. 6F depicts a schematic representation of GST-cyclin D1 wild type and ΔE mutant according to aspects of the invention;

FIG. 6G depicts MST analysis for H2B and H2BS14P in accordance with aspects of the invention;

FIG. 6H depicts a schematic representation of KDM4A fusion protein according to aspects of the invention;

FIGS. 6I and 6J are graphs of the interaction of KDM4A fusion protein in SPR with either H2BS14P or H3Kme3, respectively, in accordance with aspects of the invention;

FIG. 7A depicts a representative example of a histone array consisting of 384 unique histone modification combinations in duplicate according to aspects of the invention;

FIG. 7B is a bar graph representative of average positive intensity over average negative intensity of each modification, with the correlation between densitometric analysis of duplicate interactions for each interaction from two separate arrays shown as intensity left vs. intensity right, in accordance with aspects of the invention;

FIG. 7C depicts representative examples of histone arrays with binding shown to GST-cyclin D1, GST-ctrl, GST-ΔE, or KDM4A fusion proteins according to aspects of the invention;

FIG. 7D is a bar graph representative of average positive intensity over average negative intensity of each modification for H2B in accordance with aspects of the invention;

FIG. 7E depicts a graphical representation of binding to modified histones, in order to illustrate enhanced binding through H2B^{S14} modification according to aspects of the invention;

FIGS. 7G and 7H depict binding of cyclin D1 fusion protein to H3 modified proteins with the representative example shown, quantitated binding, and reduced binding through H2B^{S28} modification, respectively, in accordance with aspects of the invention;

FIG. 7I depicts a schematic representation illustrating the enhanced binding of cyclin D1 to modified H2B through phosphorylation at H2B^{S14P} and reduced binding to H3 upon modification by H3^{S28} according to aspects of the invention;

FIG. 8A depicts a prediction model of the cyclin D1 carboxyl terminus including the glutamate rich E region and the predicted chemical structure for a cyclin D1 pocket binding protein in accordance with aspects of the invention;

FIG. 8B depicts the effect of the compounds on HAC activity using the HCT1080 HAC reporter line according to aspects of the invention;

FIG. 9A is a bar graph showing anti-proliferation data for certain compounds in accordance with aspects of the invention; and

FIG. 9B is a model shown in stereo of cyclin D1 binding to modified histone according to aspects of the invention.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. Reference to a molecule, or to molecules, being present at a “wt. %” refers to the amount of that molecule, or molecules, present in the composition based on the total weight of the composition.

According to the present application, use of the term “about” in conjunction with a numeral value refers to a value that may be +/−5% of that numeral. As used herein, the term “substantially free” is intended to mean an amount less than about 5.0 weight %, less than 3.0 weight %, 1.0 wt. %; preferably less than about 0.5 wt. %, and more preferably less than about 0.25 wt. % of the composition.

As used herein, the term “effective amount” refers to an amount that is effective to elicit the desired biological response, including the amount of a composition that, when administered to a subject, is sufficient to achieve an effect toward the desired result. The effective amount may vary depending on the composition, the disease, and its severity and the age, weight, etc., of the subject to be treated. The effective amount can include a range of amounts. As is understood in the art, an effective amount may be in one or more doses, i.e., a single dose or multiple doses may be required to achieve the desired endpoint.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present disclosure is directed toward compositions, kits, and methods for reducing CIN, associated with chemotherapy use. In certain embodiments, the present disclosure is directed towards a method for administering a chemotherapeutic agent to a patient in need thereof. In other embodiments, the present disclosure is directed towards a method of treating, preventing, or reducing CIN resulting from administration of a chemotherapeutic agent. In other embodiments, the present disclosure is directed towards a method of reducing chemotherapy induced CIN or CIN associated with tumor progression in a patient in need thereof. In other embodiments, the present disclosure is directed towards a method of increasing survival rate or extending survival time in a patient undergoing treatment with a chemotherapeutic agent. In other embodiments, the present disclosure is directed towards a method of reducing resistance to a chemotherapeutic agent due to the development of CIN in a patient in need thereof. In other embodiments, the present disclosure is directed towards a method for reducing the CIN associated with a chemotherapy. In certain embodiments, the chemotherapeutic agent is a DNA damage inducing agent.

The present inventors have found that cyclin D1 induces CIN requiring a conserved carboxyl terminal glutamate repeat (E box region). The present inventors have surprisingly and unexpectedly discovered that administering an effective amount of cyclin D1 E domain interacting module antagonist in addition to administering an effective amount of a chemotherapeutic agent, reduces CIN activity induced by chemotherapy. For instance, the inventors surprisingly and unexpectedly discovered that aspects of the invention may increase survivorship and/or reduce the risk by reducing or eliminating one of the current problems with certain chemotherapy agents. Specifically, although numerous chemotherapeutical agents that are effective in initially killing cancer cells exist, certain chemotherapeutical agents induce CIN activity and undesirably promote tumor heterogeneity, therapy resistance, and/or cancer relapse. In view of the foregoing, the inventors surprisingly and unexpectedly discovered that administering an effective amount of cyclin D1 E domain interacting module antagonist according to some embodiments overcomes the problem of CIN activity induced by chemotherapy associated with certain chemotherapeutic agents and provides enhanced health benefits.

The enhanced health benefits of aspects of the invention may be exemplified by numerous aspects. In a first aspect, the health benefit may be to avoid increasing tumor CIN associated with administration of a chemotherapy. In another aspect, the health benefit may be to avoid increasing CIN associated with tumor growth. Other health benefits are contemplated and will be envisaged to a skilled artisan based on the present disclosure.

In accordance with a first aspect of the invention, provided is a method for treating, reducing, or ameliorating the effects of cancer. The method typically includes administering an effective amount of CIN antagonist, such as a cyclin D1 E domain interacting module antagonist (also referred hereafter as “cyclin D1 E domain antagonist”), in addition to administering an effective amount of a chemotherapeutic agent. In certain embodiments, the CIN antagonist is selected from a small molecule; an immunotherapy; siRNA/CRISPR; a gene therapy; and a combination of two or more thereof. The CIN antagonist and/or cyclin D1 E domain antagonist may be a compound according to formula I, a hydrate thereof, a solvate thereof, pharmaceutically acceptable salts thereof, an isotopic isomer thereof, a prodrug thereof, or a complex thereof:

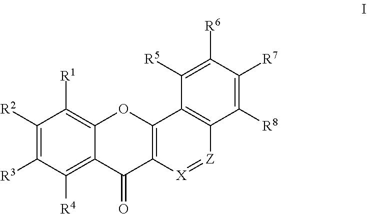

wherein,
R¹-R⁷ are independently selected from the group consisting of H, deuterium, halogen, OH, OR⁸, NO₂, CN, COOR⁸, NH₂, NR¹⁰, substituted or unsubstituted C₁₋₆ alkyl, C₁₋₆ haloalkyl, substituted or unsubstituted C₃-7 branched alkyl, and substituted or unsubstituted C₃-7 cycloalkyl, and substituted or unsubstituted aromatic ring with 0-3 heteroatoms;
R⁸ is selected from H, deuterium, C₁₋₆ alkyl, C₁₋₆ haloalkyl, C₃₋₇ branched alkyl, and C₃₋₇ cycloalkyl;
X and Z are independently selected from CH, CH₂, CO, O, CH, N, NO, and NR⁹, or together form a substituted or unsubstituted phenyl group;
R⁹ is selected from H, C₁₋₆ alkyl, C₁₋₆ haloalkyl, C₃₋₇ branched alkyl, and C₃₋₇ cycloalkyl, aromatic ring with 0-3 heteroatoms; and
R¹⁰ is selected from substituted or unsubstituted C₁₋₆ alkyl, C₁₋₆ haloalkyl, substituted or unsubstituted C₃₋₇ branched alkyl, and substituted or unsubstituted C₃₋₇ cycloalkyl, substituted or unsubstituted aromatic ring with 0-3 heteroatoms, and sulfonyl.

The compound of formula I may preferably have at least one of groups R¹-R⁷ being selected from H, D, OH, OR⁸, NO₂, CN, COOR⁸, substituted or unsubstituted C₁₋₆ alkyl, substituted or unsubstituted C₃₋₇ branched alkyl, substituted or unsubstituted C₃₋₇ cycloalkyl. In some cases, the compound of formula I has at least one of groups R¹-R⁷ being selected from substituted or unsubstituted C₁₋₆ alkyl, substituted or unsubstituted C₃₋₇ branched alkyl, and substituted or unsubstituted C₃₋₇ cycloalkyl. A plurality of groups R¹-R⁷, e.g., two, three, four, five, six, and/or seven of groups R¹-R⁷, may be H and/or D. In at least one embodiment, the compound of formula 1 includes one of groups R¹-R⁷ being selected from substituted or unsubstituted C₁₋₆ alkyl, substituted or unsubstituted C₃₋₇ branched alkyl, and substituted or unsubstituted C₃₋₇ cycloalkyl and a plurality of groups R¹-R⁷ being H and/or D. For instance, groups R¹-R⁵ and R⁷ may each be H. In one embodiment, each of groups R¹-R⁷ are H. In some embodiments, groups R¹-R⁷ do not include a halogen or an alkyl group having a halogen substituent, such as a chlorine, fluorine, Iodine, and/or bromine. In some embodiments, one or more groups R¹-R⁷ are not OH.

Additionally or alternatively, group R⁶ may be selected from H, D, NH₂, and NR¹⁰. For example, the compound of formula I may include a group R⁶ that is NH₂ or NR¹⁰. In some embodiments, a group R⁶ is H. In additional embodiments, group R⁶ is NH₂. Yet in further embodiments, group R⁶ is NR¹⁰, where R¹⁰ is selected from substituted or unsubstituted C₁₋₆ alkyl, substituted or unsubstituted C₃₋₇ branched alkyl, and substituted or unsubstituted C₃₋₇ cycloalkyl, and sulfonyl. The compounds of formula I may have a group R⁶ that is selected from NH₂. Br. and NO₂. In at least one embodiment, however, the group R⁶ of the compounds of formula I is not NO₂.

Groups X and Z of the compounds of formula I may independently be selected from CH, CH₂, CO, O, CH, N, and NR⁹. Preferably, groups X and Z are independently selected from CO, O. CH, and N. For example, one of group X or group Z may be selected from O, N, and CO, while the other of group X or group Z is selected from O, CO, CH, or CH₂. For example, group X may be N and group Z may be selected from O, CO, CH, and CH₂. In some instances, group X may be N and group Z may be selected from O, CO, and CH. The compounds of formula I may have group X selected from O or CO, while group Z is selected from N, NR⁹, and CO. In some embodiments, group X and group Z are both either CH or CO. In other embodiments, however, groups X and Z together form a phenyl group, which may be substituted or unsubstituted.

In at least one embodiment, the compound of formula I is selected from compounds of formula Ia-Ij, a hydrate thereof, a solvate thereof, pharmaceutically acceptable salts thereof, an isotopic isomer thereof, a prodrug thereof, or a complex thereof:

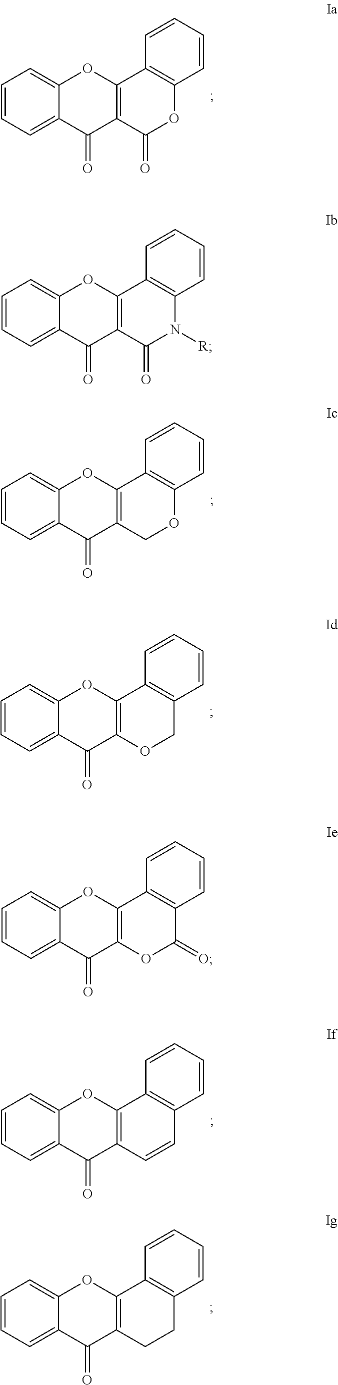

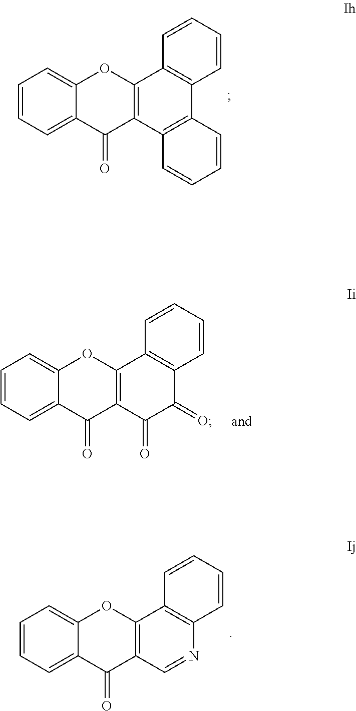 [SMILES: O=c1c2ccccc2oc2c3ccccc3c3ccccc3c12 ; O=C1C(=O)c2c(oc3ccccc3c2=O)-c2ccccc21 ; O=c1c2ccccc2oc2c1cnc1ccccc12]

In some embodiments, the compounds of formula I may have a R⁶ that is selected from NH₂, Br, and NO₂ and a Z group that is N. For instance, the compound of formula I may be selected from 2-amino-7H-chromeno[3,2-c]quinolin-7-one, N-oxide derivatives thereof, N-oxide nitro derivatives thereof, a hydrate thereof, a solvate thereof, pharmaceutically acceptable salts thereof, an isotopic isomer thereof, or a prodrug thereof.

In at least one embodiment, the compound of formula I is a compound of formula Ik-Ip, a hydrate thereof, a solvate thereof, pharmaceutically acceptable salts thereof, an isotopic isomer thereof, a prodrug thereof, or a complex thereof:

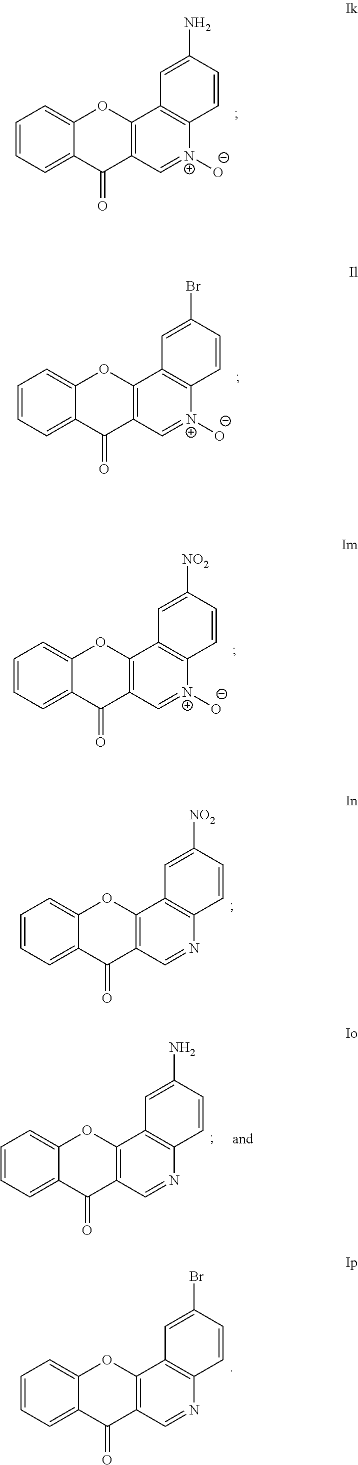 [SMILES: Nc1ccc2c(c1)c1oc3ccccc3c(=O)c1c[n+]2[O-] ; O=c1c2ccccc2oc2c1c[n+]([O-])c1ccc(Br)cc21 ; O=c1c2ccccc2oc2c1c[n+]([O-])c1ccc([N+](=O)[O-])cc21 ; O=c1c2ccccc2oc2c1cnc1ccc([N+](=O)[O-])cc12 ; Nc1ccc2ncc3c(=O)c4ccccc4oc3c2c1 ; O=c1c2ccccc2oc2c1cnc1ccc(Br)cc12]

The compound of formula I may be selected from Ik-Im, Io, and Ip, a hydrate thereof, a solvate thereof, pharmaceutically acceptable salts thereof, an isotopic isomer thereof, a prodrug thereof, or a complex thereof. In some instances, the compounds of formula I are selected from a compounds of formula Ia, Ib, Ic, Ie, I h, Ii, Ij, Ik, Il, Im, a hydrate thereof, a solvate thereof, apharmaceutically acceptable salts thereof, an isotopic isomer thereof, a prodrug thereof, a complex thereof, and a combination of two or more thereof. In some cases, it may be preferable that the compounds of formula I be selected from a compounds of formula Ia, Ib, Ie, Ih, Ii, Ik, Il, Im, a hydrate thereof, a solvate thereof, a pharmaceutically acceptable salts thereof, an isotopic isomer thereof, a prodrug thereof, a complex thereof, and a combination of two or more thereof. In further cases, the compound of formula I is selected from a compound of formula Ia, Ib, Ie, Ii, Ik, IL, Im, a hydrate thereof, a solvate thereof, a pharmaceutically acceptable salts thereof, an isotopic isomer thereof, a prodrug thereof, a complex thereof, and a combination of two or more thereof.

The present invention may be utilized with one or more chemotherapeutic agents. Various chemotherapeutic agents are well known in the art. For example, the chemotherapeutic agent may be selected from an anthracycline; a Her2 inhibitor; an immune checkpoint inhibitor; and a combination of two or more thereof. Anthracyclines are anti-neoplastic agents typically used in chemotherapy for the treatment of a wide range of circulating and solid human malignant neoplasms, including breast cancer and leukemia. In certain embodiments, the anthracycline is selected from: daunorubicin; doxorubicin; epirubicin; idarubicin; pirarubicin; valrubicin; mitoxantrone; and a combination of two or more thereof. Analogs of anthracyclines, such as mitoxantrone (trade name: Novantrone®) and vosaroxin, may in some cases be considered to be anthracycline-like compounds, and are sometimes included in the class of anthracycline chemotherapeutic agents. In at least one embodiment, the chemotherapeutic agent comprises doxorubicin.

Her2 inhibitors are typically agents that interfere with Her2 activation or function. In some cases, the Her2 inhibitor is a Her2 antibody or an antigen-binding fragment thereof, a small molecule Her2 antagonist, a Her2 tyrosine kinases inhibitor, or an antisense molecule. The Her2 antibody may inhibits Her2 ectodomain cleavage, binds to the heterodimeric binding site of Her2, and/or bind to the 4D5 epitope. In certain embodiments, the Her2 inhibitor is selected from trastuzumab; lapatinib; neratinib; pertuzumab; dacomitinib; and a combination of two or more thereof.

The immune checkpoint protein inhibitors preferably are antibodies that specifically recognize immune checkpoint proteins. In certain embodiments, the immune checkpoint inhibitor comprises a CTLA4/PD-1/PD-L1 selected from: cemiplimab; nivolumab; pembrolizumab; avelumab; durvalumab; atezolizumab; ipilimumab; and a combination of two or more thereof.

Additional immune checkpoint inhibitors include anti-PD1, anti-PDL1 or anti-CTLA4 antibodies. Examples of PD-1 inhibitors include without limitation humanized antibodies blocking human PD-1, such as lambrolizumab, pidilizumab, and nivolumab. Other PD-1 inhibitors may include presentations of soluble PD-1 ligand including without limitation PD-L2 Fc fusion protein also known as B7-DC-Ig or AMP-244. In addition, immune checkpoint inhibitors may include without limitation humanized or fully human antibodies blocking PD-L, such as MEDI-4736 (disclosed in WO2011066389 A1, which is incorporated herein in its entirety for all purposes), MPDL3280A (disclosed in U.S. Pat. No. 8,217,149 B, which is incorporated herein in its entirety for all purposes).

Other immune checkpoint inhibitors include those used to stimulate immune responses. Such immune checkpoint inhibitors include inhibitors that directly or indirectly stimulate or enhance antigen-specific T-lymphocytes. For example, the immune checkpoint inhibitors may be agents targeting immune checkpoint proteins and pathways involving PD-L2, LAG3, BTLA, B7H4 and TIM3. Any of the foregoing immune checkpoint protein inhibitors may be used as such or analogues thereof may be used; for example, chimerized, humanized or human forms of the immune check point inhibitor antibodies.

Histone deacetylase (HDAC) inhibitors typically have the ability to induce growth arrest, differentiation, and/or cell necrosis of tumor cells. Examples of HDAC inhibitors include, but are not limited to, hydroxamic acid derivatives, short chain fatty acids (SCFA), cyclic tetrapeptides, benzamide derivatives, or electrophilic ketone derivatives. Non-limiting examples of HDAC inhibitors include: A) m-carboxycinnamic acid bishydroxyxamide (CBHA), trichostatin A (TSA), trichostatin C, salicylic hydroxamic acid, azelaic bishydroxyxamic acid (ABHA), azelaic-1-hydroxyxamate-9-anilide (AAHA), 6-(3-chlorophenylureido) carpoic hydroxamic acid (3C1-UCHA), oxamplatin, A-161906, scriptide, PXD-101, LAQ-824, CHAP, Hydroxamic acid derivatives selected from MW2796, and MW2996; B) Trapoxin A (TPX)-cyclic tetrapeptide (cyclo-(L-phenylalanyl-L-phenylalanyl-D-pipecolinyl-L-2-amino-8-oxo-9,10-epoxy Decanoyl); FR901228 (FK 228, depoxypeptide); FR225497 cyclic tetrapeptide; apisidine cyclic tetrapeptide [cyclo (NO-methyl-L-tryptophanyl-L-isorucinyl-D-pipecoli Nyl-L-2-amino-8-oxodecanoyl)]; Apicid Ia, Apicid Ib, Apicid Ic, Apicid Ila, and Apicid IIb; CHAP, HC-toxin cyclic tetra peptide; is selected from cyclic and Cloud Mai dosin; WF27082 cyclic Tetrapeptide; C) sodium butyrate, isovalerate, valerate, 4-phenylbutyrate (4-PBA), phenylbutyrate (PB), propionate, butyramide, isobutyramide, phenylacetate, 3-bromopropionate, Short chain fatty acid (SCFA) selected from tributyrin, valproic acid and valproate and Pivanex™; D) CI-994, MS-27-275 (MS-275) [N-(2-aminophenyl)-4-[N-(pyridin-3-ylmethoxycarbonyl) aminomethyl]benzamide] and MS-benzamide derivative is selected from 3′-amino derivative of 27-275; E) electrophilic ketone derivatives selected from trifluoromethyl ketones and a-ketoamides such as N-methyl-a-ketoamides; and F) other HDAC inhibitors, including natural products, psammaplin and defudecin. In at least one embodiment, the HDAC inhibitor is such as subveroylanilide hydroxamic acid (SAHA).

BET inhibitors typically interfere with BET protein interactions via bromodomain inhibition, resulting in modulation of transcriptional programs that are often associated with diseases characterized by dysregulation of cell cycle control. The BET inhibitor may bind to the bromodomain and/or extraterminal (BET) proteins BRD2, BRD3, BRD114 and BRDT, preventing protein-protein interaction between BET proteins and acetylated histones and transcription. Non-limiting examples of BET inhibitors include JQ1, GSK525762A and OTX-015. The term “JQ1” refers to a compound of formula:

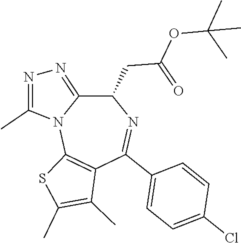 [SMILES: Cc1sc2c(c1C)C(c1ccc(Cl)cc1)=N[C@@H](CC(=O)OC(C)(C)C)c1nnc(C)n1-2]

The term “GSK525762A” refers to a compound of formula:

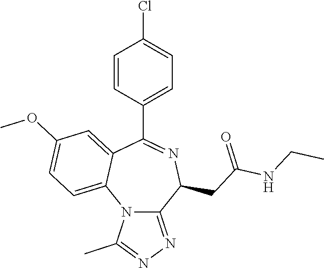 [SMILES: CCNC(=O)C[C@@H]1N=C(c2ccc(Cl)cc2)c2cc(OC)ccc2-n2c(C)nnc21]

The term “OTX-015” refers to a compound of formula:

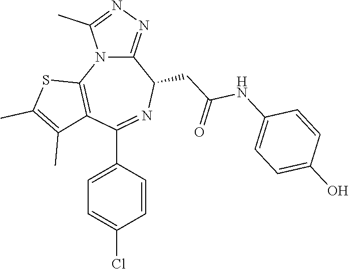 [SMILES: Cc1sc2c(c1C)C(c1ccc(Cl)cc1)=N[C@@H](CC(=O)Nc1ccc(O)cc1)c1nnc(C)n1-2]

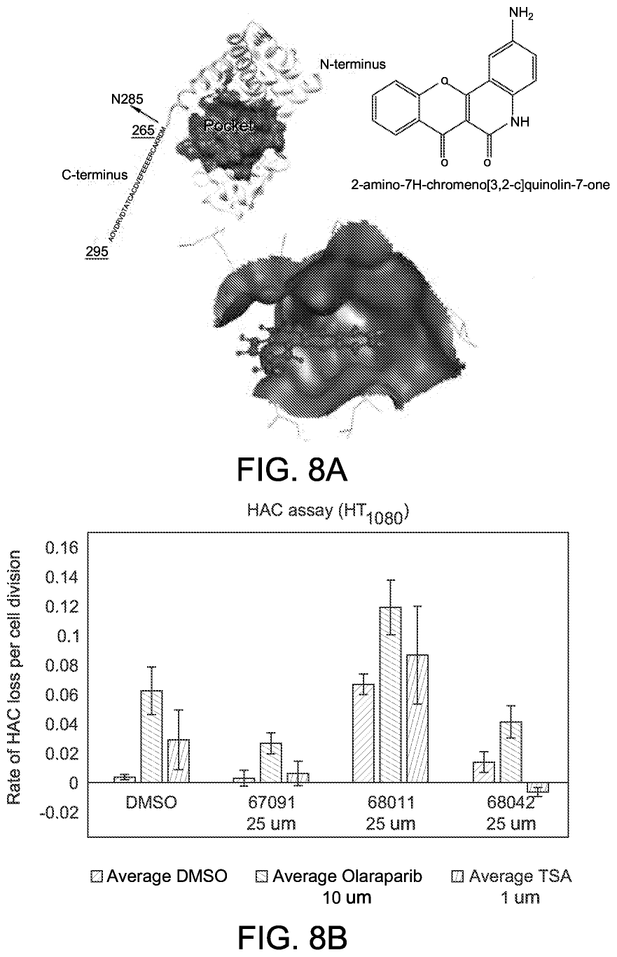

Poly-(ADP-ribose) polymerases (PARPs) inhibitors are enzymes involved in DNA-damage repair. Dose limiting side effects of PARPi are very serious and include gastrointestinal symptoms, anemia, and hematopoietic compromise. The PARP inhibitor may be a NAD-dependent or a NAD-independent PARP inhibitor. Examples of PARP inhibitors include niparib, olaparib, niraparib, rucparib, veliparib, BMN 673, CEP 9722, MK 4827, E 7016, 4-iodo-3-nitrobenzamide, benzamide, a metabolite thereof, or any combination of two or more thereof. In at least one embodiment, the NAD-dependent inhibitor is F502 and/or MC240022. Additional description of PARP inhibitors may be found in U.S. Pat. Nos. 7,732,491, 8,894,989, U.S. Patent Publication No. 2020/0129476, and U.S. Patent Publication No. 2015/0344968, all of which are incorporated herein by reference in their entirety for all purposes. In at least one embodiment, the chemotherapy agent comprises Olaparib (see FIG. 8B).

Other chemotherapy agents that may be used with the aspects of present invention, and may benefit from such aspects of the present invention, include, e.g., carboplatin, cisplatin, cyclophosphamide, docetaxel, erlotinib, etoposide, fluorouracil, gemcitabine, imatinib mesylate, irinotecan, methotrexate, paclitaxel, sorafmib, sunitinib, topotecan, vincristine or vinblastine, and others. It is also contemplated that any conventional therapeutic agents may be used together with the present disclosure, and may benefit from the present disclosure for use in treating cancer, or cancer metastasis.

Without being bound by theory, it is believed that exposure of cancer cells to certain DNA-damaging agent results in sufficient DNA damage to trigger the DNA damage response and temporary S phase arrest to allow for DNA repair. The DNA damage response is believed to be regulated by two homologous protein kinases, ataxia telangiectasia (ATM) and ataxia telangiectasia Rad3-related (ATR). ATR signals to regulate DNA replication, cell cycle transitions, and DNA repair through the phosphorylation of hundreds of substrates, including checkpoint kinase 1 (Chkl). DNA damage induces apoptosis of cells and is widely believed to be the major antiproliferative mechanism of DNA damaging anticancer drugs.

In one aspect, the present disclosure therefore provides a method for administering a chemotherapeutic agent to a patient in need thereof comprising administering an effective amount of a CIN antagonist (e.g., cyclin D1 E domain antagonist) followed by administering an effective amount of a chemotherapeutic agent. The methods disclosed herein may utilize any of the CIN antagonists (e.g., cyclin D1 E domain antagonists) and/or the chemotherapeutic agents disclosed herein. In further embodiments, the present disclosure provides for a method of treating, preventing, or ameliorating a symptom associated with CIN, including tumor heterogeneity and therapy resistance, resulting from the administration of a chemotherapeutic agent comprising administering an effective amount of a CIN antagonist followed by administering an effective amount of a chemotherapeutic agent. In other embodiments, the present disclosure provides for a method reducing CIN in a patient in need thereof comprising administering an effective amount of a CIN antagonist followed by administering an effective amount of a chemotherapeutic agent. In yet other embodiments, the present disclosure provides for a method of increasing survival rate or extending survival time in a patient undergoing treatment with a chemotherapeutic agent comprising administering an effective amount of a CIN antagonist followed by administering an effective amount of a chemotherapeutic agent. In yet other embodiments, the present disclosure provides for a method of reducing the effective dose of a chemotherapeutic agent in a patient in need thereof comprising administering an effective amount of a CIN antagonist followed by administering an effective amount of a chemotherapeutic agent. In other embodiments, the present disclosure provides for method for reducing the CIN associated with a chemotherapy, comprising co-administering an effective amount of a CIN antagonist and a chemotherapeutic agent.

In certain embodiments, the CIN antagonist is administered prior to the administration of the chemotherapeutic agent. In other embodiments, the CIN antagonist is co-administered with the administration of the chemotherapeutic agent. In various embodiments, the CIN antagonist and chemotherapeutic agent are administered contemporaneously. In certain embodiments, the CIN antagonist is administered prior to the chemotherapeutic agent. In certain embodiments, the CIN antagonist may be administered from about 1 minute to about 72 hours prior to administration of the chemotherapeutic agent, optionally about 15 minutes, or 30 minutes, or 60 minutes, 90 minutes, or 2 hours, or 4 hours, or 8 hours, or 12 hours, or 18 hours, or 24 hours, or 36 hours, or 48 hours, or 60 hours or 72 hours, prior to administration of the chemotherapeutic agent. In further embodiments, in addition to a contemporaneous CIN antagonist and chemotherapeutic agent administration or a CIN antagonist administration prior to the chemotherapeutic agent administration, further step comprising administering an additional dose of a CIN antagonist following administration of the chemotherapeutic agent may be performed.

In one aspect, the present disclosure therefore provides for a composition comprising an effective amount of a CIN antagonist and an effective amount of a chemotherapeutic agent. In other embodiments, the present disclosure therefore provides for composition comprising an effective amount of doxorubicin; an effective amount of lapatinib and/or rapamycin; and a pharmaceutically acceptable carrier.

According to another aspect of the invention, provided is a kit. The kit may include a CIN antagonist; optionally, a chemotherapeutic agent; and instructions for the administration of each. In some embodiments, the kits may also include one or more composition(s). For example, the kit may include a pharmaceutical composition comprising a CIN antagonist and, optionally, a chemotherapeutic agent. Preferably, the kit includes an effective amount of, or a composition comprising an effective of, a CIN antagonist and, optionally, a chemotherapeutic agent.

As used herein “therapeutically effective amount” or “therapeutically effective dosage” refers to an amount that is effective to achieve a desired therapeutic result, such as reducing chemotherapy induced CIN or CIN associated with tumor progression in a patient in need thereof. In some embodiments, the desired therapeutic result is a reduction in the growth of cancer cells and/or reduction in the likelihood of metastasizing. For example, a therapeutically effective amount of a compound (e.g., a CIN antagonist or a chemotherapeutic agent) may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day.

In some cases, the amount of a CIN antagonist, a chemotherapeutic agent, a hydrate thereof, a solvate thereof, pharmaceutically acceptable salts thereof, an isotopic isomer thereof, a prodrug thereof, a complex thereof, or a combination of two or more thereof present in composition is more than about 1 μg. For example, the composition may comprise an amount of a CIN antagonist, a chemotherapeutic agent, a hydrate thereof, a solvate thereof, pharmaceutically acceptable salts thereof, an isotopic isomer thereof, a prodrug thereof, a complex thereof, or a combination of two or more thereof of about 2 μg or more, about 5 μg or more, about 10 μg or more, about 100 μg or more, about 500 μg or more, about 1000 μg or more, about 1500 μg or more, about 2000 μg or more, about 2500 μg or more, about 3000 μg or more, about 3500 μg or more, about 4000 μg or more, about 4500 μg or more, about 5000 μg or more, about 5500 μg or more, about 6000 μg or more, about 6500 μg or more, about 7000 μg or more, about 7500 μg or more, about 8000 μg or more, about 8500 μg or more, about 9000 μg or more, about 9500 μg or more, about 10 mg or more, about 20 mg or more, about 30 mg or more, about 40 mg or more, about 50 mg or more, about 60 mg or more, about 70 mg or more, about 80 mg or more, about 90 mg or more, about 100 mg or more, about 150 mg or more, about 200 mg or more, about 250 mg or more, about 300 mg or more, about 350 mg or more, about 400 mg or more, about 450 mg or more, about 500 mg or more, about 550 mg or more, about 600 mg or more, about 650 mg or more, about 700 mg or more, about 800 mg or more, about 900 mg or more, or about 1 g or more. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference.

Additionally or alternatively, the amount of CIN antagonist, chemotherapeutic agent, a hydrate thereof, a solvate thereof, pharmaceutically acceptable salts thereof, an isotopic isomer thereof, a prodrug thereof, a complex thereof, or a combination of two or more thereof present in the composition may be about 0.01 wt. % to about 95 wt. %, about 0.1 wt. % to about 95 wt. %, about 1 wt. % to about 95 wt. %, about 5 wt. % to about 95 wt. %, about 10 wt. % to about 95 wt. %, about 15 wt. % to about 95 wt. %, about 20 wt. % to about 95 wt. %, about 30 wt. % to about 95 wt. %, about 40 wt. % to about 95 wt. %, about 50 wt. % to about 95 wt. %, about 60 wt. % to about 95 wt. %, about 70 wt. % to about 95 wt. %, about 80 wt. % to about 95 wt. %; about 0.01 wt. % to about 85 wt. %, about 0.1 wt. % to about 85 wt. %, about 1 wt. % to about 85 wt. %, about 5 wt. % to about 85 wt. %, about 10 wt. % to about 85 wt. %, about 15 wt. % to about 85 wt. %, about 20 wt. % to about 85 wt. %, about 30 wt. % to about 85 wt. %, about 40 wt. % to about 85 wt. %, about 50 wt. % to about 85 wt. %, about 60 wt. % to about 85 wt. %, about 70 wt. % to about 85 wt. %; about 0.01 wt. % to about 75 wt. % about 0.1 wt. % to about 75 wt. %, about 1 wt. % to about 75 wt. %, about 5 wt. % to about 75 wt. %, about 10 wt. % to about 75 wt. %, about 15 wt. % to about 75 wt. %, about 20 wt. % to about 75 wt. %, about 30 wt. % to about 75 wt. %, about 40 wt. % to about 75 wt. %, about 50 wt. % to about 75 wt. %, about 60 wt. % to about 75 wt. %; about 0.01 wt. % to about 65 wt. %, about 0.1 wt. % to about 65 wt. %, about 1 wt. % to about 65 wt. %, about 5 wt. % to about 65 wt. %, about 10 wt. % to about 65 wt. %, about 15 wt. % to about 65 wt. %, about 20 wt. % to about 65 wt. %, about 30 wt. % to about 65 wt. %, about 40 wt. % to about 65 wt. %, about 50 wt. % to about 65 wt. %; about 0.01 wt. % to about 55 wt. %, about 0.1 wt. % to about 55 wt. %, about 1 wt. % to about 55 wt. %, about 5 wt. % to about 55 wt. %, about 10 wt. % to about 55 wt. %, about 15 wt. % to about 55 wt. %, about 20 wt. % to about 55 wt. %, about 30 wt. % to about 55 wt. %, about 40 wt. % to about 55 wt. %; about 0.01 wt. % to about 45 wt. %, about 0.1 wt. % to about 45 wt. %, about 1 wt. % to about 45 wt. %, about 5 wt. % to about 45 wt. %, about 10 wt. % to about 45 wt. %, about 15 wt. % to about 45 wt. %, about 20 wt. % to about 45 wt. %, about 30 wt. % to about 45 wt. %; about 0.01 wt. % to about 35 wt. %, about 0.1 wt. % to about 35 wt. %, about 1 wt. % to about 35 wt. %, about 5 wt. % to about 35 wt. %, about 10 wt. % to about 35 wt. %, about 15 wt. % to about 35 wt. %, about 20 wt. % to about 35 wt. %; about 0.01 wt. % to about 25 wt. %, about 0.1 wt. % to about 25 wt. %, about 1 wt. % to about 25 wt. %, about 5 wt. % to about 25 wt. %, about 10 wt. % to about 25 wt. %; about 0.1 wt. % to about 15 wt. %, about 1 wt. % to about 15 wt. %, about 5 wt. % to about 15 wt. %, about 10 wt. % to about 15 wt. %; about 0.01 wt. % to about 25 wt. %, about 0.1 wt. % to about 10 wt. %, about 1 wt. % to about 10 wt. %, or about 5 wt. % to about 10 wt. %, including ranges and subranges thereof, based on the total weight of the composition.

Additionally or alternatively, the compositions may be formulated to have a weight ratio of CIN antagonist to chemotherapeutic agent (or therapeutically effective amounts thereof) of 1:100 to 100:1, 1:50 to 50:1, 1:20 to 20:1 1:10 to 10:1, 1:9 to 10:1, 1:8 to 10:1, 1:7 to 10:1, 1:6 to 10:1, 1:5 to 10:1, 1:4 to 10:1, 1:3 to 10:1, 1:2 to 10:1, 1:1 to 10:1, 1:10 to 9:1, 1:10 to 8:1, 1:10 to 7:1, 1:10 to 6:1, 1:10 to 5:1, 1:10 to 4:1, 1:10 to 3:1, 1:10 to 2:1, or 1:10 to 1:1, including ranges and subranges thereof. One of ordinary skill would be able to prepare the compositions disclosed herein using known methods and/or in the art in view of the disclosure herein.

The compositions typically further comprise at least one excipient. Suitable excipients include pharmaceutically acceptable excipients, such as diluents, binders, fillers, buffering agents, pH modifying agents, disintegrants, dispersants, preservatives, lubricants, taste-masking agents, flavoring agents, coloring agents, or combinations thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, and saccharides.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl pa Imitate, citric acid, sodium citrate; chelators such as EDTA or EGTA; and antimicrobials, such as parabens, chlorobutanol, or phenol.

In a further embodiment, the excipient may be a lubricant. Nonlimiting examples of suitable lubricants include minerals such as talc or silica; and fats such as vegetable stearin, magnesium stearate, or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

The composition may be formulated into various dosage forms and administered by a number of different means that will deliver a therapeutically effective amount of the active ingredient. Such compositions may be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term, “parenteral,” as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, the composition may be a food supplement or a cosmetic.

Solid dosage forms for oral administration may be contained in capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, suspensions, or creams as generally known in the art.

In certain embodiments, a composition may comprise a compound that is encapsulated in a suitable vehicle to either aid in the delivery of such compound to target cells, to increase the stability of the composition, or to minimize potential toxicity of the composition. As will be appreciated by a skilled artisan, a variety of vehicles are suitable for delivering a composition of the present invention. Non-limiting examples of structured fluid delivery systems may include nanoparticles, liposomes, microemulsions, micelles, dendrimers, and other phospholipid-containing systems. Methods of incorporating compositions into delivery vehicles are known in the art. Disclosures relating to the administration of compositions using nanotechnology and/or nano drug delivery systems are described in U.S. Pat. No. 7,491,407, U.S. Patent Publication No. 2013/0225412, U.S. Pat. No. 9,180,102, which are all incorporated herein by reference in their entirety for all purposes.

In one alternative embodiment, a liposome delivery vehicle may be utilized. Liposomes, depending upon the embodiment, may be used for delivery of a composition a CIN antagonist, a chemotherapeutic agent, a hydrate thereof, a solvate thereof, pharmaceutically acceptable salts thereof, an isotopic isomer thereof, a prodrug thereof, a complex thereof, or a combination of two or more thereof in view of their structural and chemical properties. Generally, liposomes are spherical vesicles with a phospholipid bilayer membrane. The lipid bilayer of a liposome may fuse with other bilayers (e.g., the cell membrane), thus delivering the contents of the liposome to cells.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soybeans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1, 1 ‘-dioctadecyl-3,3,3’,3′-tetramethylindocarbocyanine perchloarate, 3,3′-deheptyloxacarbocyanine iodide, 1,T-dedodecyl-3,3,3′,3′-tetramethylindocarbocyanine perchloarate, 1,T-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetro nitrile, alcohols, dimethylformamide, tetrahydrofuran, or combinations thereof.

Liposomes carrying a composition disclosed herein may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211, and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration, and composition of lipid, concentration of multivalent cations, rate of mixing, presence of and concentration of solvent.

The composition may be formulated as part of a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids.

Any of the phospholipids detailed above for liposomes are suitable for embodiments directed to microemulsions. A composition comprising at least one anti-viral therapeutic derivative may be encapsulated in a microemulsion by any method generally known in the art.

In yet another embodiment, the composition may contain compounds delivered in a dendritic macromolecule, or a dendrimer. Generally, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

The methods of the disclosure may include administering an amount of a composition topically, orally, or parenterally. For oral administration, the method may include administering an amount of the composition in the form of a solid dosage or a liquid dosage. Solid dosage forms for oral administration include capsules, tablets, caplets, pills, powders, pellets, and granules. Liquid dosages of the composition may be in the form of aqueous suspensions, elixirs, or syrups. For these, the composition may be combined with various sweetening or flavoring agents, coloring agents, and, if so desired, emulsifying and/or suspending agents, as well as diluents such as water, ethanol, glycerin, and combinations thereof.

For parenteral administration, the dosage of composition may be an aqueous solution, an oil-based solution, or in the form of a solid dosage. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. In some instances, parental administration may be subcutaneous, intravenous, intramuscular, or intrasternal injection, or infusion.

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

“Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

The term “alkyl” and “alkylene” as used herein refer to straight- and branched-chain hydrocarbon groups, preferably containing one to sixteen carbon atoms. Examples of alkyl groups are C₁₋₄alkyl groups. As used herein the designation C_{x-y}, wherein x and y are integers, denotes a group having from x to y carbons, e.g., a C₁₋₄alkyl group is an alkyl group having one to four carbon atoms. Nonlimiting examples of alkyl groups include, but are not limited to, methyl, ethyl, n-propyl, isopropyl, n-butyl, sec-butyl (2-methylpropyl), t-butyl (1,1-dimethylethyl), and the like. Nonlimiting examples of alkylene groups include methylene (—CH₂—) and ethylene (—CH₂CH₂—).

The term “cycloalkyl” as used herein refers to an aliphatic cyclic hydrocarbon group, preferably containing three to eight carbon atoms. Nonlimiting examples of cycloalkyl groups include cyclopropyl, cyclobutyl, cyclopentyl, cyclohexyl, and the like.

The terms “substituted alkyl,” “substituted cycloalkyl,” and “substituted alkylene” as used herein refer to an alkyl, cycloalkyl, or alkylene group having one or more substituents. The substituents include, but are not limited to, cycloalkyl, aryl, heteroaryl, heterocycloalkyl, substituted aryl, substituted heteroaryl, substituted heterocycloalkyl, N(R^{d})₂, OR^{d}, SR^{d}, sulfoxide, sulfonyl, halo, carboxyl, acyl, carboxy, hydrazino, hydrazono, and hydroxyamino. The preferred substituted alkyl groups have one to four carbon atoms, not including carbon atoms of the substituent group. Preferably, a substituted alkyl group is mono- or di-substituted at one, two, or three carbon atoms. The substituents can be bound to the same carbon or different carbon atoms.

The term “alkoxy” as used herein refers to a straight- or branched-chain alkyl, optionally substituted, group attached to the parent molecule through an oxygen atom, typically by a carbon to oxygen bond, i.e., —OR, wherein R is an alkyl group. The hydrocarbon group of the alkoxy group preferably contains one to four carbon atoms. Typical alkoxy groups include, but are not limited to, methoxy, ethoxy, n-propoxy, isopropoxy, n-butoxy, sec-butoxy, t-butoxy, and the like. The term “thioalkoxy” is similarly defined, except sulfur replaces oxygen.

The term “acyl” as used herein refers to a R^{e}C(═O) group attached to the parent molecule through a carbonyl (C═O) group. R^{e} is selected from the group consisting of alkyl, substituted alkyl, cycloalkyl, substituted cycloalkyl, aryl, substituted aryl, heteroaryl, substituted heteroaryl, heterocycloalkyl, and substituted heterocycloalkyl groups.

The term, “aryl” as used herein refers to monocyclic, fused bicyclic, and fused tricyclic carbocyclic aromatic ring systems including, but not limited to, phenyl, naphthyl, tetrahydronaphthyl, phenanthrenyl, biphenylenyl, indanyl, indenyl, anthracenyl, fluorenyl, and the like.

The term “heteroaryl” as used herein refers to monocyclic, fused bicyclic, and fused tricyclic aromatic ring systems, wherein one to four-ring atoms are selected from the group consisting of oxygen, nitrogen, and sulfur, and the remaining ring atoms are carbon, said ring system being joined to the remainder of the molecule by any of the ring atoms. Nonlimiting examples of heteroaryl groups include, but are not limited to, pyridyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, thiophenyl, furanyl, quinolinyl, isoquinolinyl, benzoxazolyl, benzimidazolyl, benzothiazolyl, and the like.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl groups ring systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuryl, and the like.

The terms “substituted aryl.” “substituted heteroaryl,” and “substituted heterocycloalkyl” as used herein refer to an aryl, heteroaryl, or heterocycloalkyl group substituted by a replacement of one, two, or three of the hydrogen atoms thereon with a substitute selected from the group consisting of halo, OR^{d}, N(R^{d})₂, C(═O)N(R⁴)₂, CN, alkyl, substituted alkyl, mercapto, nitro, aldehyde, carboxy, carboxyl, carboxamide, aryl, substituted aryl, heteroaryl, substituted heteroaryl, cycloalkyl, substituted cycloalkyl, heterocycloalkyl, substituted heterocycloalkyl, O(CH₂)₁₋₃N(R^{d})₂, O(CH₂)₁₋₃CO₂H, and trifluoromethyl.

The term “aldehyde” as used herein refers to a —CHO group.

The term “amino” as used herein refers an —NH₂ or —NH— group, wherein each hydrogen in each formula can be replaced with an alkyl, cycloalkyl, aryl, heteroaryl, heterocycloalkyl, substituted alkyl, substituted cycloalkyl, substituted aryl, substituted heteroaryl, or substituted heterocycloalkyl group, i.e., N(R^{e})₂. In the case of —NH₂, the hydrogen atoms also can be replaced with substituents taken together to form a 5- or 6-membered aromatic or nonaromatic ring, wherein one or two carbons of the ring optionally are replaced with a heteroatom selected from the group consisting of sulfur, oxygen, and nitrogen. The ring also optionally can be substituted with an alkyl group. Examples of rings formed by substituents taken together with the nitrogen atom include, but are not limited to, morpholinyl, phenylpiperazinyl, imidazolyl, pyrrolidinyl, (N-methyl)piperazinyl, piperidinyl, and the like.

The term “carbamoyl” as used herein refers to a group of the formula —NR^{d}C(═O)R^{d}, —OC(═O)N(R^{d})₂, and —NR^{d}C(=O)—, wherein R^{d} is defined above.

The term “carbonyl” as used herein refers to a CO, C(O), or C(═O) group.

The term “carboxyl” as used herein refers to —CO₂H.

The term “carboxy” as used herein refers to a —COOR^{d}, wherein R^{d} is defined above.

The term “carboxamide” as used herein refers to —C(═O)N(R^{g})₂, wherein R^{g} is defined as hydro, alkyl, substituted alkyl, aryl, substituted aryl, heteroaryl, substituted heteroaryl, heterocycloalkyl, substituted heterocycloalkyl, cycloalkyl, substituted cycloalkyl, or OR^{d}, or the R^{g} groups are taken together with the nitrogen to which they are attached to form a five- or six-membered optionally substituted aromatic or nonaromatic ring, wherein one or two carbons of the ring optionally are replaced with a heteroatom selected from the group consisting of sulfur, oxygen, and nitrogen.

The term “thiocarboxamide” as used herein, refers to —C(═S)N(R⁸)₂, wherein Reis defined above.

The term “mercapto” as used herein refers to —SR^{d}, wherein R^{d} is defined above.

The term “sulfonamido” as used herein refers to —NHSO₂R_{g}, wherein R^{g} is defined above.

The term “cyano” as used herein refers to a —C≡N group, also designated —CN.

The term “hydroxyamino” acs used herein refers to a —NHOH group.

The term “hydrazono” as used herein refers to a —N—NH₂ group, wherein one or both hydrogen atoms can be replaced with an alkyl or substituted alkyl group.

The terms “trifluoromethyl” and “trifluoromethoxy” as used herein refer to —CF₃ and OCF₃, respectively.

The term “halo” as used herein refers to bromo, chloro, iodo, and fluoro.

The term “sulfonyl” as used herein refers to group represented by —SO₂— or —SO₂R^{d}, wherein R^{d} is defined above.

The term “sulfamyl” as used herein refers to —SO₂N(R^{g})₂, wherein R^{g} is defined above. The term “sulfo” as used herein refers to —SO₃H.

The term “nitro” as used herein refers to —NO₂.

In the structures herein, for a bond lacking a substituent, the substituent is methyl, for example,

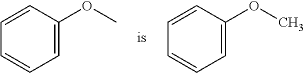

When no substituent is indicated as attached to a carbon atom on a ring, it is understood that the carbon atom contains the appropriate number of hydrogen atoms. In addition, when no substituent is indicated as attached to a carbonyl group or a nitrogen atom, for example, the substituent is understood to be hydrogen, e.g.,

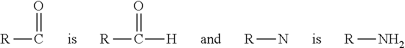

The abbreviation “Me” is methyl and Bn is benzyl.

The notation N(R^{x})₂, wherein x represents an alpha or numeric character, such as, for example, R^{d} is used to denote two R^{x} groups attached to a common nitrogen atom. When used in such notation, the R^{x} group can be the same or different, and is selected from the group as defined by the R^{x} group

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

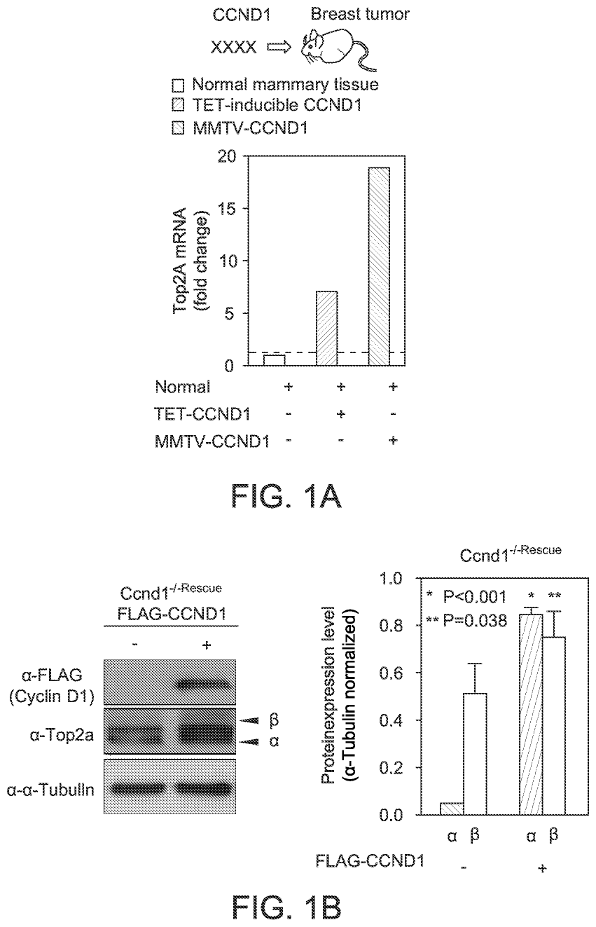

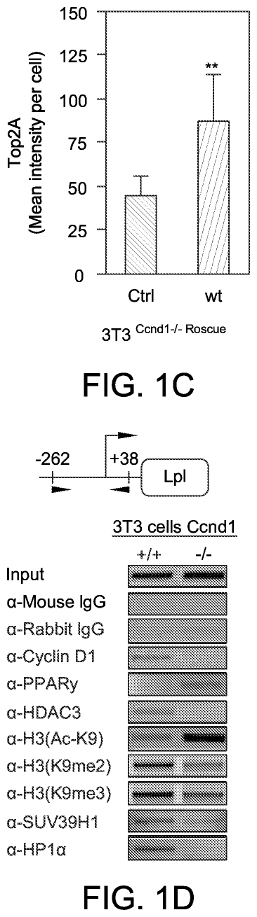

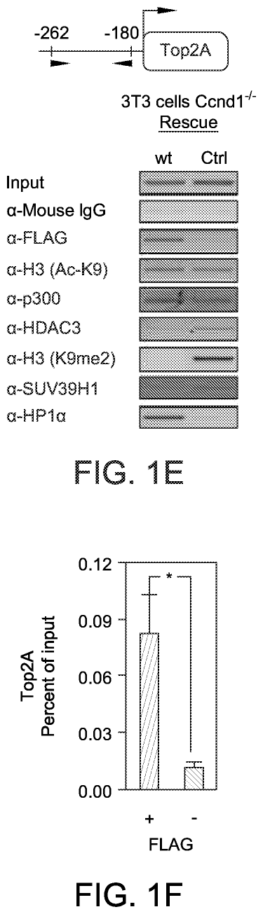

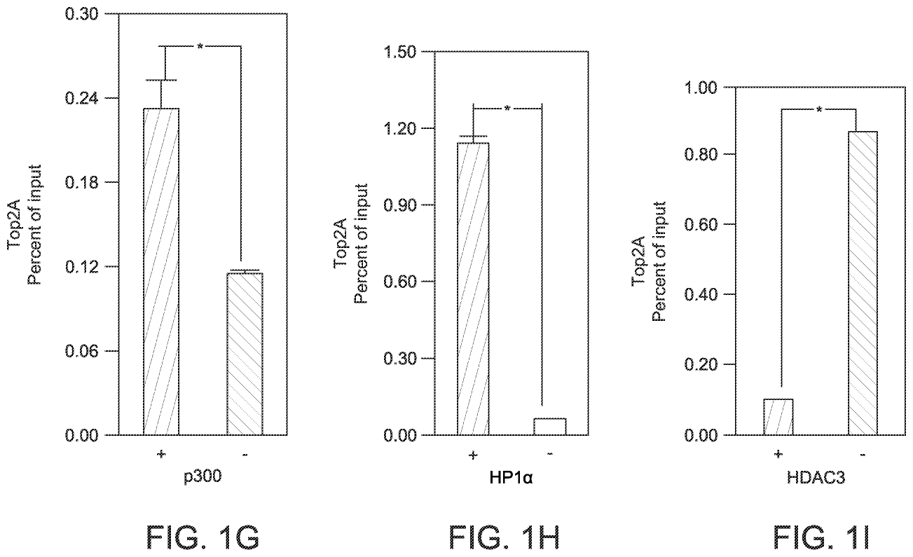

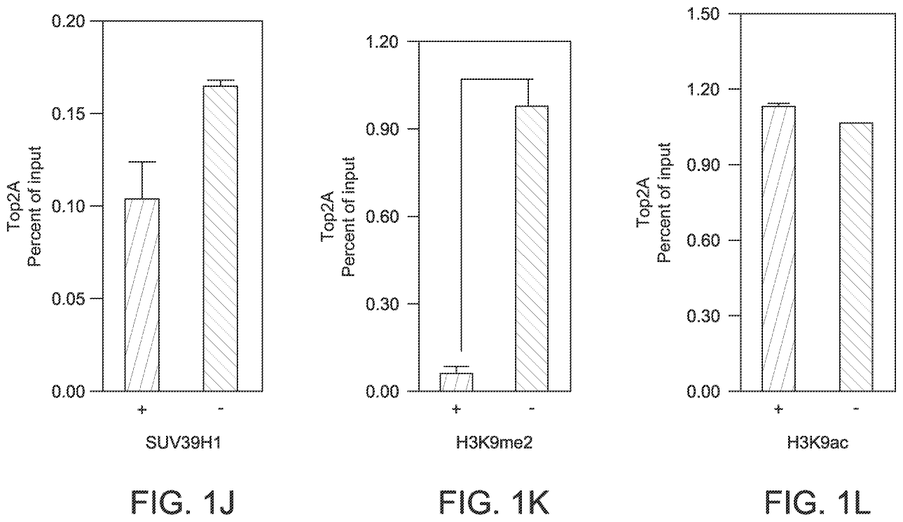

Transgenic mice were obtained having either Tet-inducible cyclin D1 mammary epithelial cell (7 days of transgenic induction) or MMTV-cyclin D1 transgenic induced mammary gland tumors. As cyclin D1 regulates expression of target genes governing CIN (e.g., Top2A, Aurkb, BRCA1, CENPE), the expression of such target genes governing CIN in the transgenic mice was assessed. As seen in FIG. 1A, the transgenic mice had a Top2A mRNA abundance in the mammary gland of Tet-inducible cyclin D1 mammary epithelial cell and MMTV-cyclin D1 transgenic induced mammary gland tumors. The abundance of Top2a and B tubulin in cyclin D1^{-/-} 3T3 cells rescued with a cyclin D1 expression vector with quantitation of multiplicate experiments was determined, as seen in the Western blot of FIG. 1B. Top2a intensity was assessed by immunofluorescent staining in cyclin D1^{-/-} cyclin D1 rescue 3T3 cells with data shown as mean±SEM for N=5, as seen in FIG. 1C. Endogenous murine LPL ChIP assay was produced based on the transgenic mice (see FIG. 1D). Top2A ChIP assays were produced with antibodies directed to target proteins-namely, α-mouse IgG, α-FLAG, α-H3 (Ac-K9), α-p300, α-HDAC3, α-H3 (K9mc2), α-SUV39H1, and α-HP1α (see FIG. 1E-1L). The ChIP assays were assessed using real-time quantitative PCR (qPCR), with all data being mean±SEM and n=3 experiments. P-values were determined by the student's t-test.

### Example 2

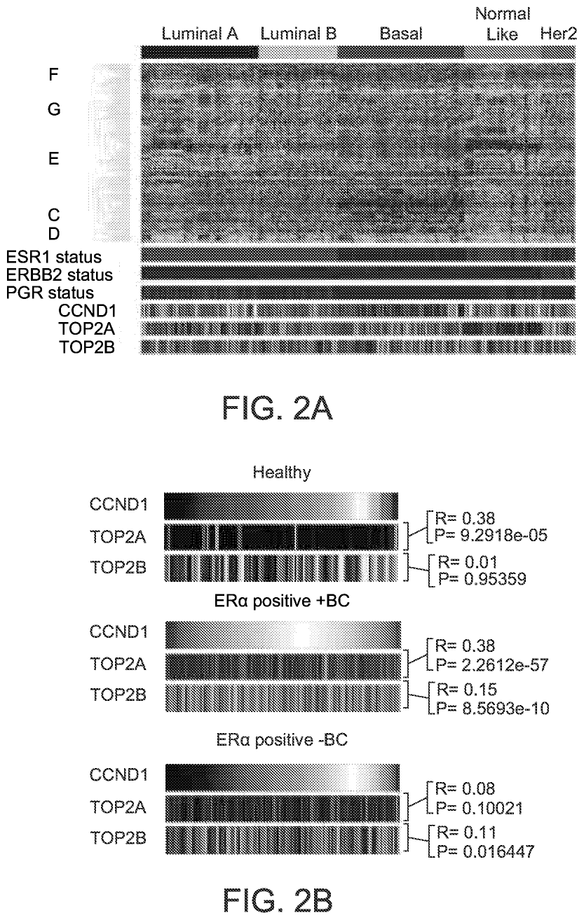

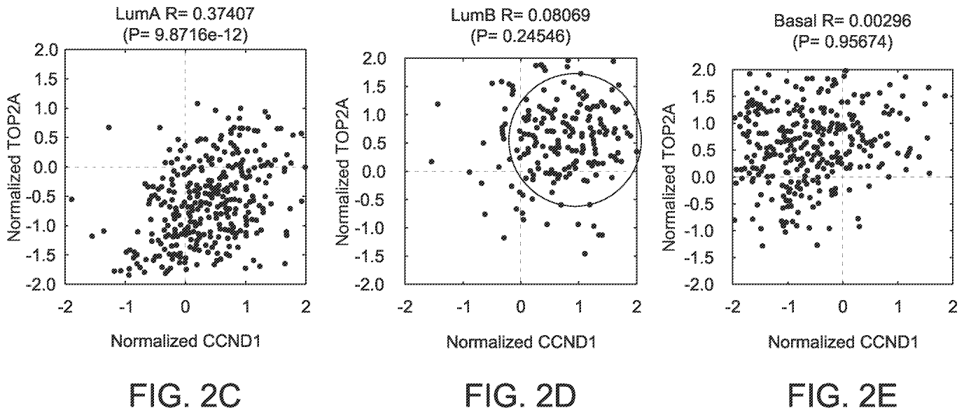

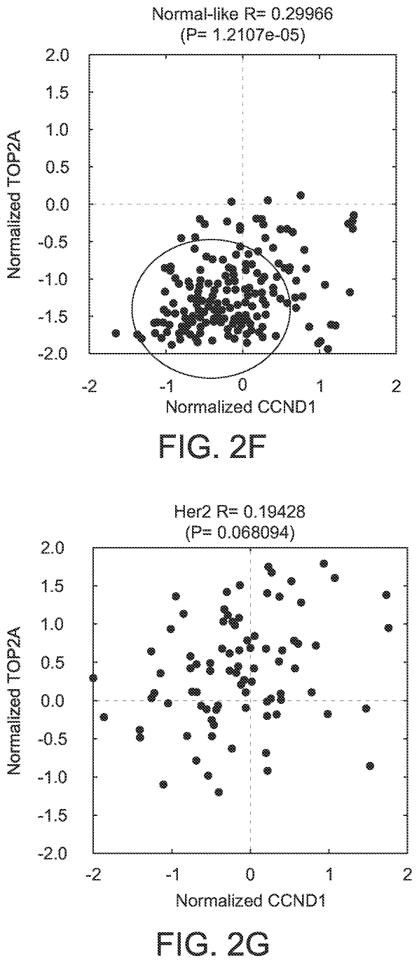

FIG. 2A depicts a heatmap depiction of samples from combined breast cancer microarray datasets that were assigned to the five breast cancer microarray subtypes. Specifically, the five breast cancer microarray subtypes included the predicted estrogen receptor α (ESR1), epidermal growth factor receptor ERBB2, progesterone receptor (PGR), together with TOP2A and TOP2B expression, with cyclin D1 (CCND1) expression level across the 5 subtypes. FIG. 2B depicts relative enrichment of TOP2A in ERα⁺ vs. ERα⁻ breast cancer. FIGS. 2C-2G are scatter plots depicting CCND1 transcript level versus TOP2A, showing the relationship between TOP2A and cyclin D1 expression in luminal B and normal-like subtype-specific (as indicated by the circle in FIGS. 2F and 2D). The circle highlights luminal B and normal like subtype in which TOP2A and cyclin D1 expression level show a co-segregation. Overall, FIGS. 2A-2G indicate there is a significant correlation between cyclin D1 and TOP2A in patients samples, which provides supportive evidence that cyclin D1 induces TOP2A in addition to the evidence shown in FIGS. 1A-1L that cyclin D1 induces TOP2A expression in tissue culture and in the mammary gland in the mouse model.

### Example 3

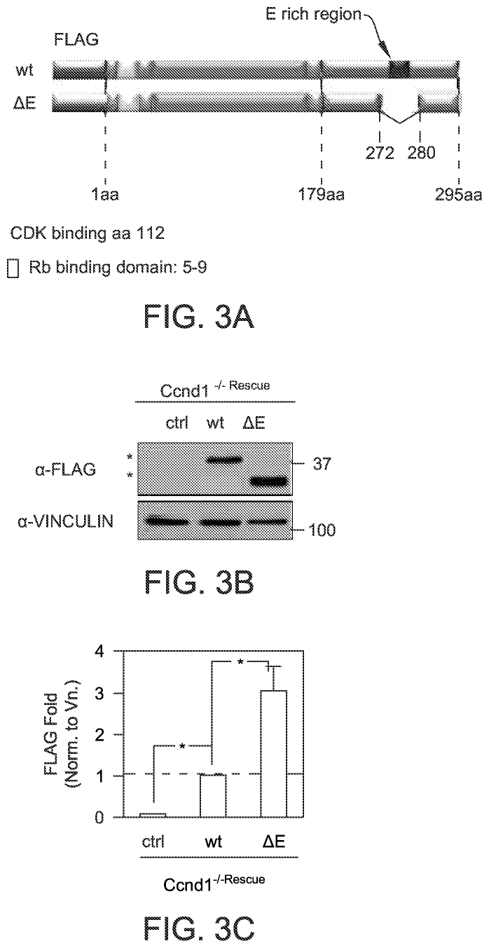

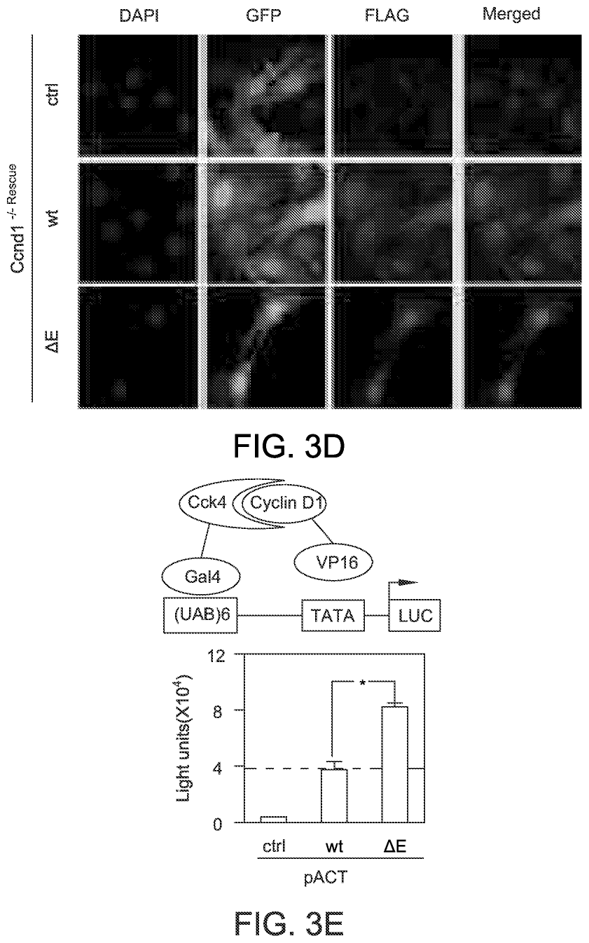

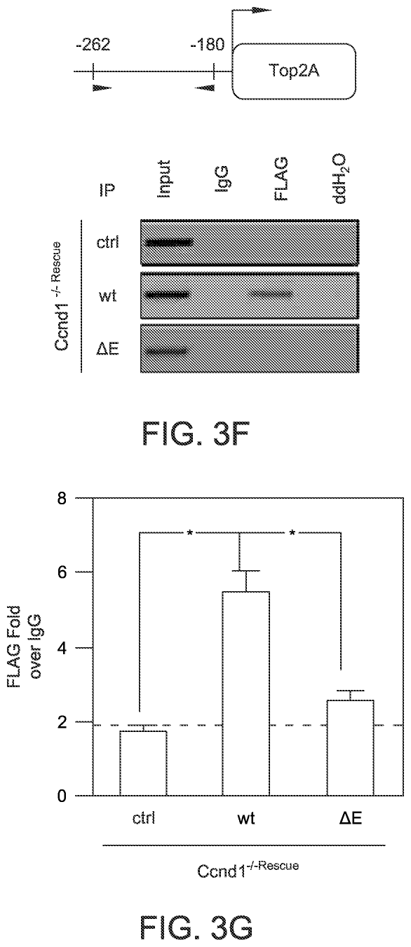

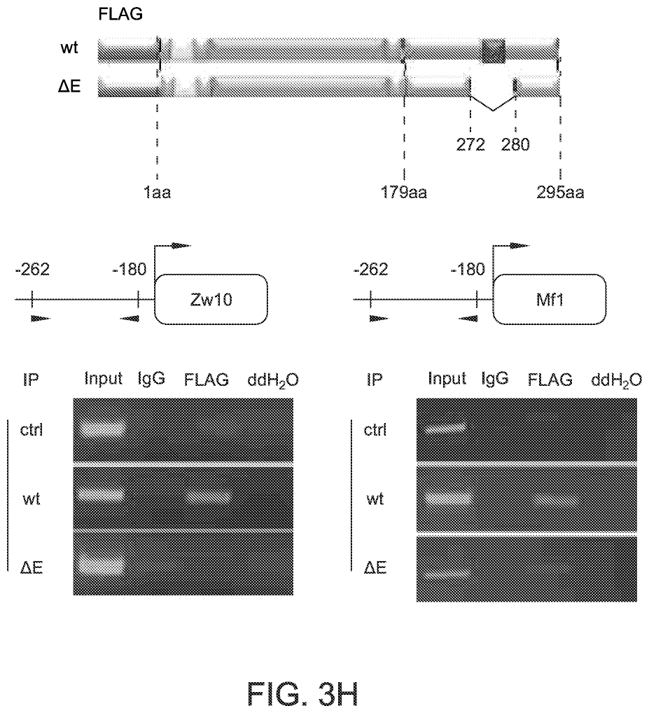

Cyclin D1 and its binding site to two additional genes, which were identified as being induced in the chromosomal signature by cyclin D1, were evaluated to determine the region of the bind site. A schematic representation of cyclin D1^{wt} and cyclin D1^{ΔE} mutant is shown in FIG. 3A. As seen in FIG. 3B, a Western blot detection was prepared for the FLAG-tagged expression vectors. FIG. 3C depicts a densitometric analysis of cyclin D1 proteins, shown as mean±SEM for N=5 separate experiments (P<0.05). FIG. 3D depicts immunofluorescence staining of the FLAG epitope, with GFP stain (Green) from the vector IRES, DAPI stain (Blue) and FLAG stain (Red). FIG. 3E depicts Mammalian 2-hybrid interaction of cyclin D1^{wt} and cyclin D1^{ΔE}, shown as mean±SEM for N>5 separate transfections. FIG. 3F depicts ChIP assays of the FLAG-tagged cyclin D1 proteins. Occupancy was detected by PCR and FIG. 3D depicts qPCR (mean±SEM for N=3. FIG. 3H depicts a schematic representation of cyclin D1^{wt} and cyclin D1^{ΔE} with representative ChIP analysis of the cyclin D1 binding site at two additional genes that are induced by cyclin D1 and identified within the chromosomal instability signature (Zw10, Mlf1).

### Example 4

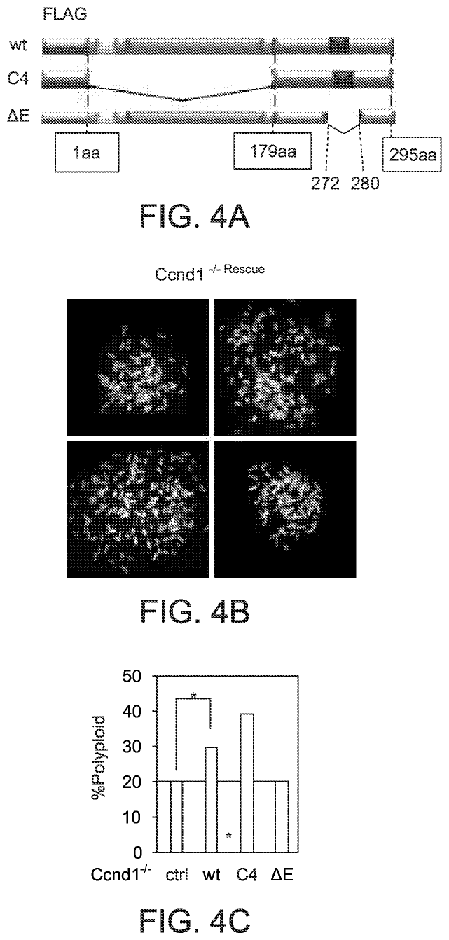

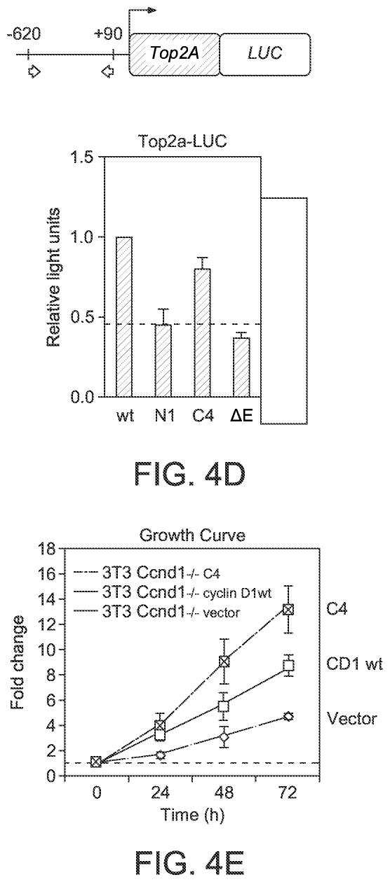

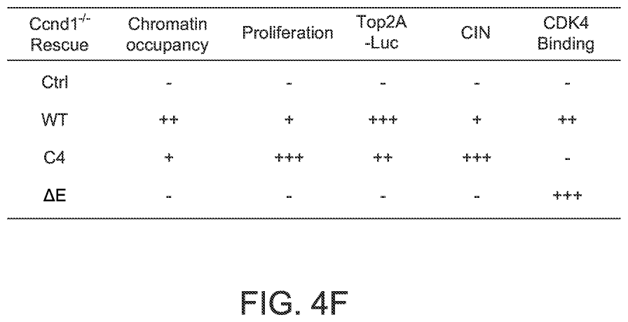

Cyclin D1^{wt} and mutants were evaluated to assess chromatin occupancy, proliferation, Top2A-Luc, CIN, and CDK4 Binding, as seen in FIGS. 4A-4F. The mutational analysis was conducted to identify the minimal region necessary for the induction of CIN. As shown below, the identified region is the E domain. FIG. 4A depicts a schematic representation of cyclin D1^{wt} and mutants. A Karyotype was determined through SKY analysis for cyclin D1^{-/-} cells rescued with either cyclin D1^{wt} or cyclin D1 mutants (see FIG. 4B). The mean polyploidy of cyclin D1^{-/-} cells rescued with either cyclin D1^{wt} or cyclin D1 mutants is depicted in FIG. 4C. FIG. 4D depicts Top2A promoter luciferase reporter assays shown as mean±SEM for N>5 separate transfections. FIG. 4E depicts cellular proliferation assays conducted in cyclin D1^{-/-} 3T3 cells, rescued either with cyclin D1^{wt}, mutant or Control vector. FIG. 4F depicts a summary of cyclin D1 mutant properties.

### Example 5

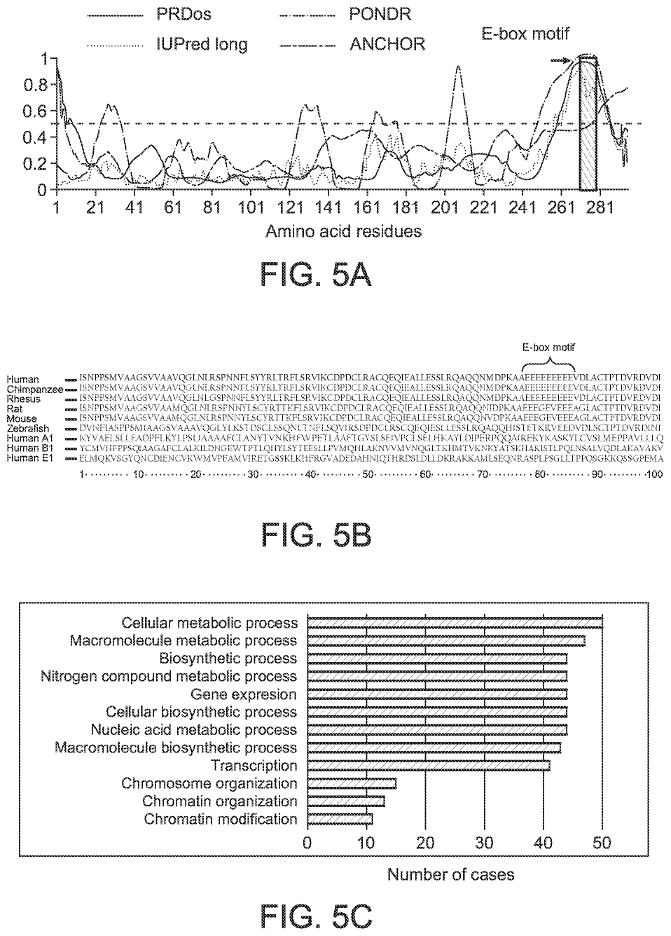

Cyclin D1 was evaluated to determine the structure and domains of Cyclin D1. FIG. 5A depicts a series of computer models that define the structure of the E region of Cyclin D1. It was determined that the E region, which is believed to induce CIN has an intrinsically disorder structure and has a high degree of unfoldedness (also referred to as “super unfolded”). For instance, the bigger the number on the y-axis of FIG. 5A, the higher the degree of unfoldedness. More specifically, the predictions show that the carboxy-terminal 40 residues (aa256-aa295) have a strong tendency to be intrinsically disordered, whereas ANCHOR suggests that last 15 amino acids contain a protein-protein interaction site. A multiple sequence alignment of the last (carboxyl-terminal) 100 amino acids of cyclin D1 from various species and of human cyclin A1, B1 and E1 was determined, as seen in FIG. 5B. FIG. 5B reveals that region E of cyclin D1 is conserved between species, which supports the idea that this new region is biologically relevant. An E-box motif was found in 149 human proteins by BLASTP, and their enrichment in GO Biological Process (BP) categories (“regulation of . . . ”, number of hits in the given category/149 polyE proteins) is shown in FIG. 5C. Notably, the E-box motif is conserved between species and is in the middle of the region required for the induction of chromosomal instability. This motif is also found in 149 other genes in the human genome that have functions related to chromosomal function and metabolism. Thus, without being limited to any particular theory, it is believed that the discovered region E of cyclin D is of broad importance in other genes.

### Example 6

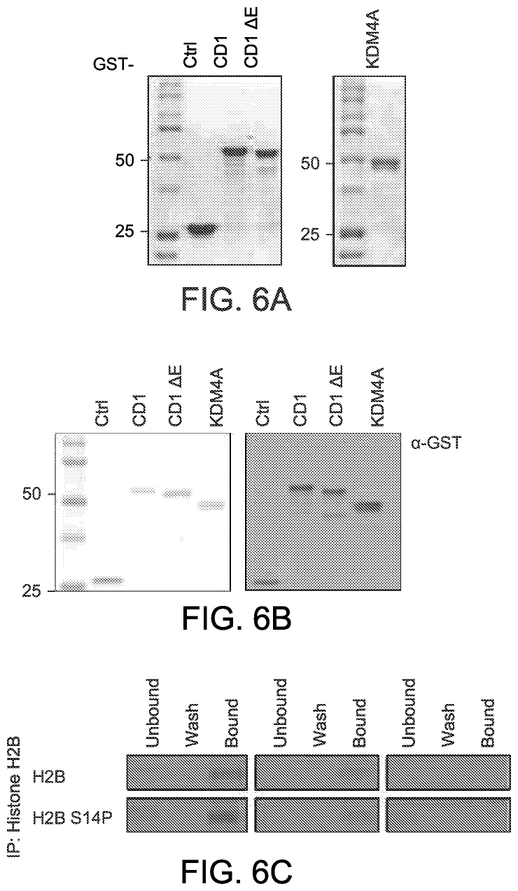

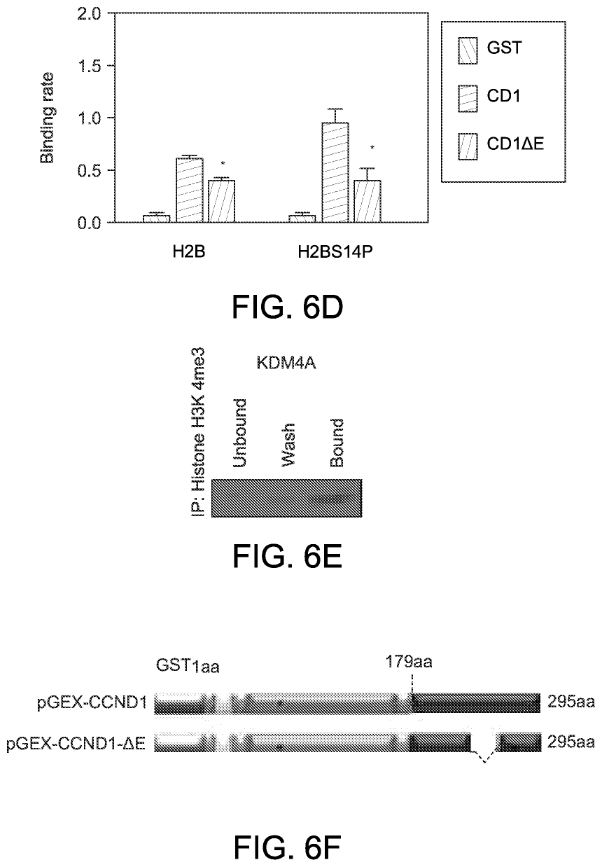

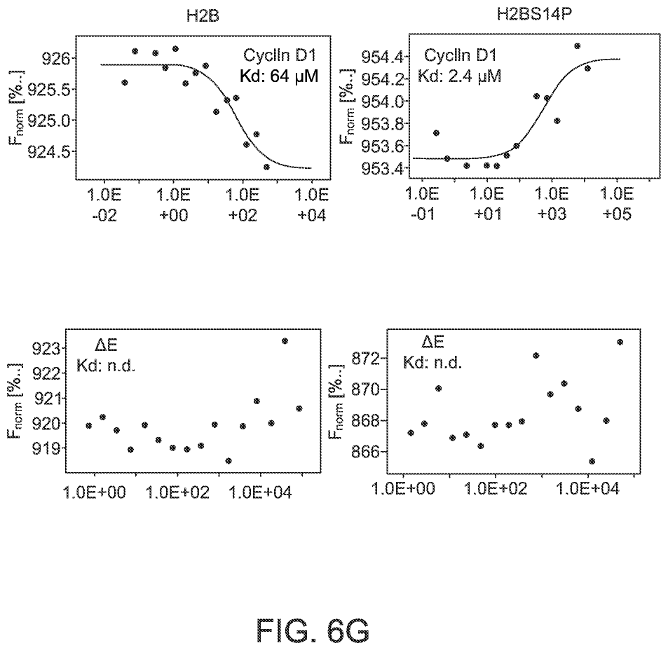

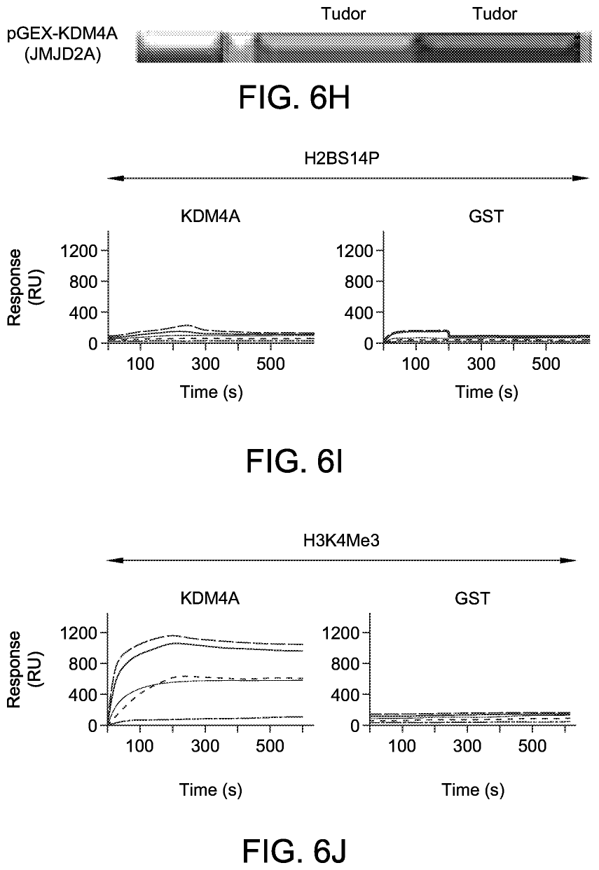

A Coomassie staining (see FIG. 6A) and a Western blot (see FIG. 6B) for cyclin D1 (CD1), cyclin D1 E-box deleted (CD1 □E) and KDM4A GST fusion proteins were produced. A GST fusion protein pull-down technique was employed (see FIG. 6C) and quantified as shown in FIG. 6D, with the mean±SEM for multiplicate experiments for GST-CD1, GST-CD1ΔE or GST-ctrl fusion proteins with histone peptide (H2B) and S14-phosphorylated histone peptide (H2BS14P). In comparison, a GST fusion protein pull-down of histone H3K4me3 with KDM4A is depicted in FIG. 6E. FIG. 6F depicts schematic representation of GST-cyclin D1 wild type and ΔE mutant with FIG. 6G depicting MST analysis with H2B and H2BS14P, indicating determined Kd, or the lack of binding (Kd cannot be determined, n.d.). FIG. 6H depicts a schematic representation of KDM4A fusion protein and interaction in SPR with either H2BS14P (see FIG. 6I) or H3Kme3 (see FIG. 6J). Overall, FIGS. 6A-6B show that the proteins were produced and were of the correct molecular weight. The proteins were then used to determine and assess binding to histones and modified histones, as shown in FIGS. 6C-6J.

### Example 7

The interaction of cyclin D1 fusion with the histones of DNA was evaluated. The identification of the domain E of cyclin D1 was unexpected and surprising as it was unknown that Cyclin D1 had an intrinsically disordered domain. As discussed in the foregoing Examples, the existence of the intrinsically disordered domain was identified by the mutational deletion analysis of cyclin D1 and the unbiased in silico computer modeling.

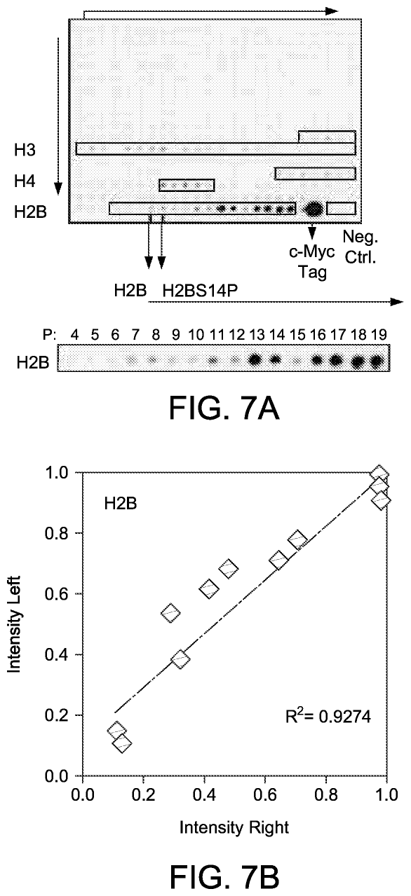

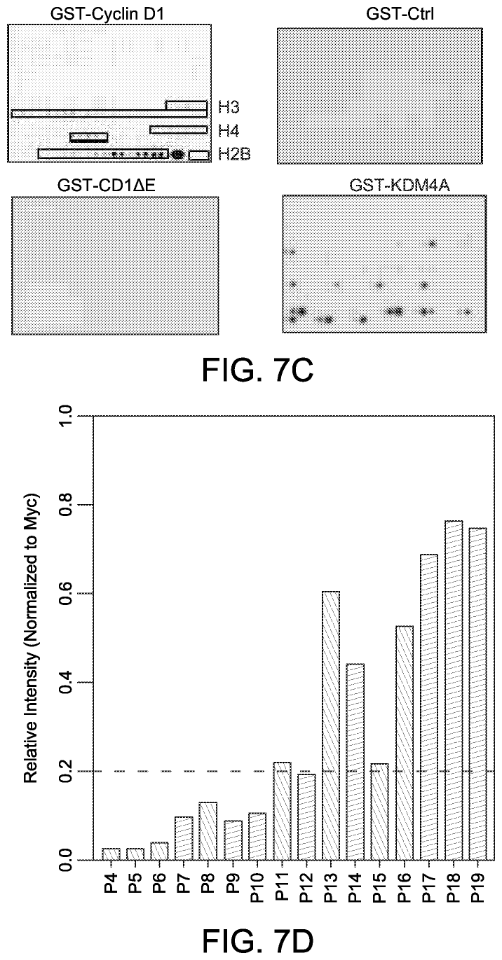

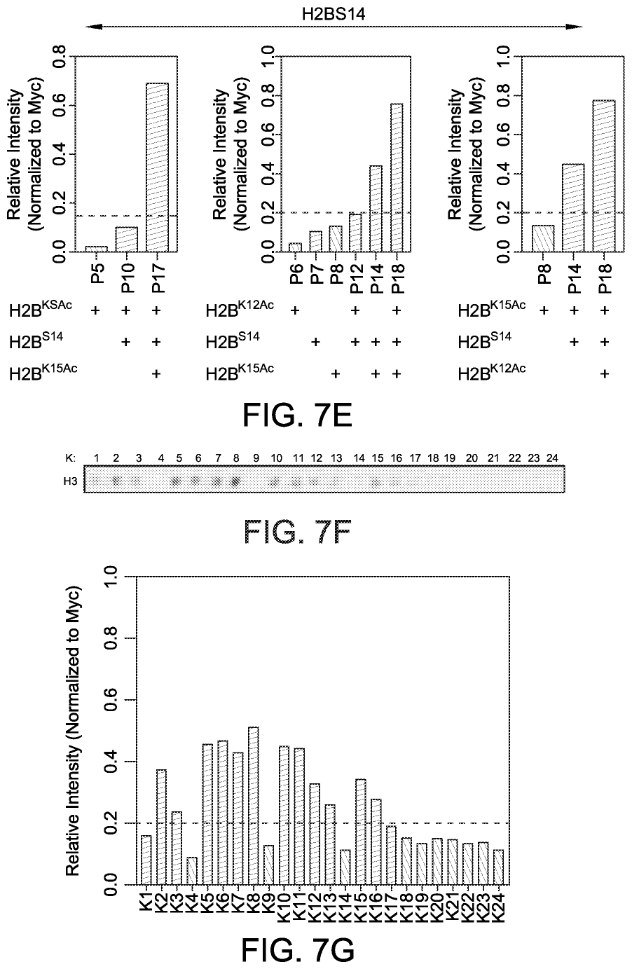

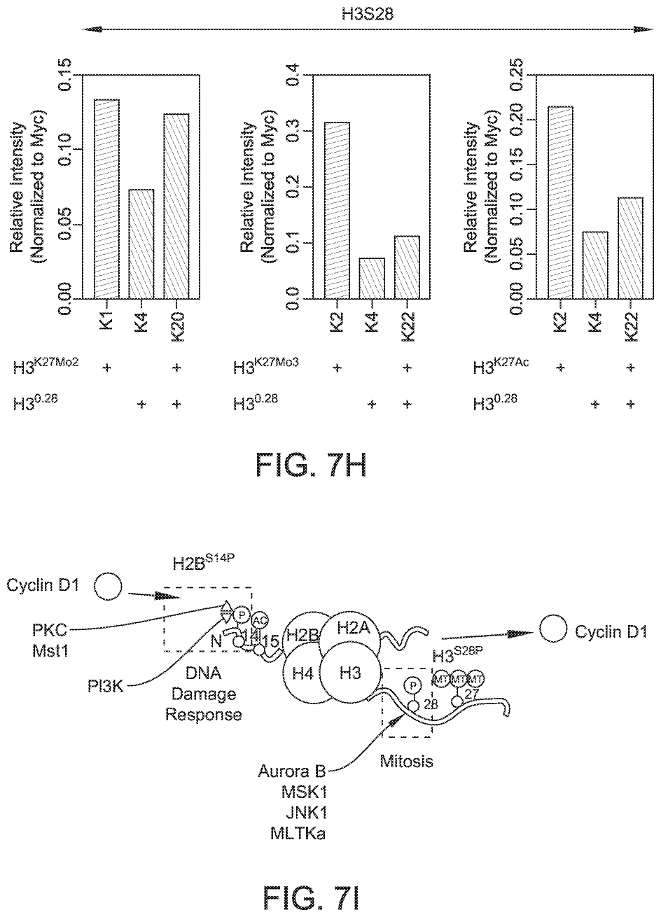

FIG. 7A depicts a representative example of a histone array consisting of 384 unique histone modification combinations in duplicate. The array includes up to four separate modifications on the same 19mer peptide. As each spot is arrayed in duplicate, representative examples of cyclin D1 binding to modified histones were tested by histone-modification arrays and analyzed by active Motif software. FIG. 7B depicts binding representative of average positive intensity over average negative intensity of each modification. The correlation between densitometric analysis of duplicate interactions for each interaction from two separate arrays is shown as intensity left vs. intensity right. Notably, the intensity is highly reproducible between duplicate binding assessments (R²=0.927). FIG. 7C depicts representative examples of histone arrays (384 unique histone modification on the same 19mer peptide), with binding shown to GST-cyclin D1, GST-ctrl, GST-ΔE, or KDM4A fusion proteins. FIG. 7D depicts representative and quantitated binding, representative of average positive intensity over average negative intensity of each modification for H2B. FIG. 7E depicts a graphical representation of binding to modified histones, in order to illustrate enhanced binding through H2B$14 modification. FIGS. 7G and 7H depict binding of cyclin D1 fusion protein to H3 modified proteins with representative example, quantitated binding, and reduced binding through H2B^{S28} modification.

The enhanced binding with modified histone suggests that the cyclin D1 intrinsically disordered domain binds modified histones. Furthermore, these studies show the cyclin D1 intrinsically disordered domain region binds with higher affinity to modified histones than unmodified histones. FIG. 7I depicts a schematic representation illustrating the enhanced binding of cyclin D1 to modified H2B through phosphorylation at H2B^{S14P} and reduced binding to H3 upon modification by H3^{S28}.

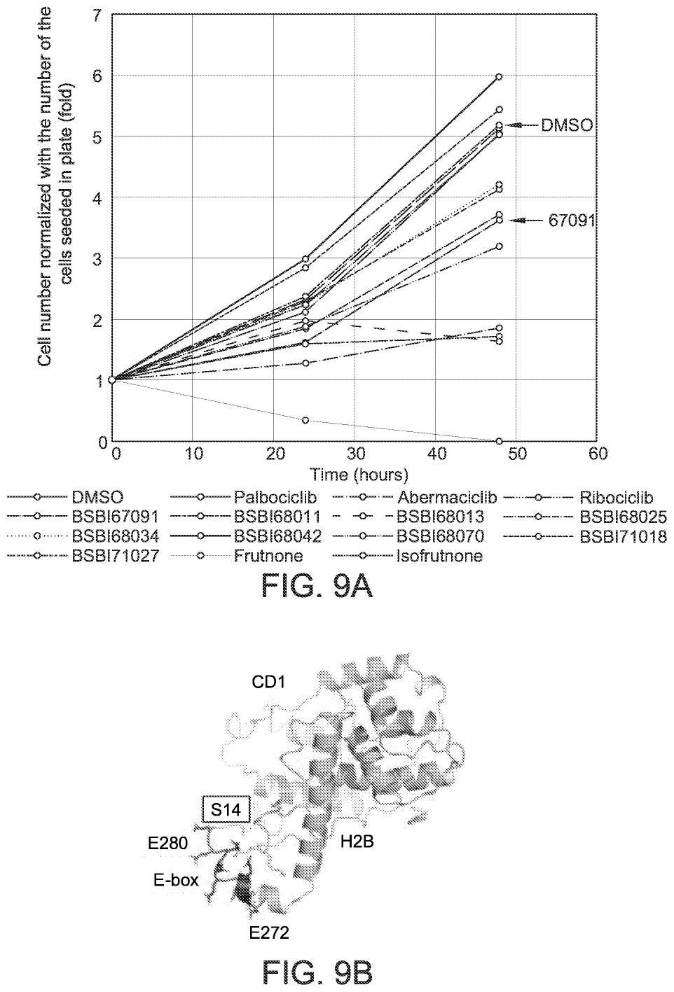

FIG. 8A depicts a prediction model of the cyclin D1 carboxyl terminus including the glutamate rich E region and the predicted chemical structure for a cyclin D1 pocket binding protein 2-amino-7H-chromeno[3,2-c]quinolin-7-one. FIG. 8B depicts the effect of the compounds on HAC activity using the HCT1080 HAC reporter line. FIG. 9A provides a bar graph showing anti-proliferation data for certain compounds. In FIG. 9A BSBI67091 refers to 2-bromo-7H-chromeno[3,2-c]quinolin-7-one; BSBI78034 refers to 2-bromo-7-oxo-7H-chromeno[3,2-c]quinoline 5-oxide; BSBI71027 refers to 2-amino-6-hydroxy-7H-chromeno[3,2-c]quinolin-7-one; BSBI68011 refers to 2-amino-7H-chromeno[3,2-c]quinolin-7-one; BSBI68042 refers to 2-amino-7-oxo-7H-chromeno[3,2-c]quinoline 5-oxide; BSBI68013 refers to 2-nitro-7H-chromeno[3,2-c]quinolin-7-one; BSBI68070 refers 2-bromo-5H-chromeno[3,2-c]quinoline-6,7-dione; BSBI68025 refers to 2-nitro-7-oxo-7H-chromeno[3,2-c]quinoline 5-oxide; andBSBI71018 refers to 2-bromo-6-hydroxy-7H-chromeno[3,2-c]quinolin-7-one.

### Example 9

Another model cyclin D1 binding to modified histone (H2B^{S14P}) using αfold was prepared, which identified in an unbiased interrogation, using multiple distinct peptide lengths (19-24 amino acids) for H3^{S14P}, the binding of H3^{S14P} to the cyclin D1 E box region (IDD domain) (shown as a stereo TIF in FIG. 9B).

1-73. (canceled)

74. A method for reducing the chromosomal instability associated with a chemotherapy, comprising co-administering an effective amount of a CIN antagonist and a chemotherapeutic agent.

75. The method according to claim 74, wherein the CIN antagonist and chemotherapeutic agent are administered contemporaneously.

76. The method according to claim 74, wherein the CIN antagonist is administered prior to the chemotherapeutic agent.

77. The method according to claim 74, wherein the chemotherapeutic agent is a DNA damage inducing agent.

78. The method according to claim 77, wherein the CIN antagonist is administered from about 1 minute to about 72 hours prior to administration of the chemotherapeutic agent, optionally about 15 minutes, or 30 minutes, or 60 minutes, 90 minutes, or 2 hours, or 4 hours, or 8 hours, or 12 hours, or 18 hours, or 24 hours, or 36 hours, or 48 hours, or 60 hours or 72 hours, prior to administration of the chemotherapeutic agent.

79. The method according to claim 77, further comprising the step of administering an additional dose of a CIN antagonist following administration of the chemotherapeutic agent.

80. The method according to claim 74, wherein the chemotherapeutic agent is selected from: an anthracycline; a Her2 inhibitor; an immune checkpoint inhibitor; and a combination of two or more thereof.

81. The method according to claim 74, wherein:
the anthracycline is selected from: daunorubicin; doxorubicin; epirubicin; idarubicin; valrubicin; mitoxantrone; and a combination of two or more thereof;
the Her2 inhibitor is selected from: trastuzumab; lapatinib; neratinib; pertuzumab; dacomitinib; and a combination of two or more thereof; and
the immune checkpoint inhibitor comprises a CTLA4/PD-1/PD-L1 selected from: cemiplimab; nivolumab; pembrolizumab; avelumab; durvalumab; atezolizumab; ipilimumab; and a combination of two or more thereof.

82. The method according to claim 74, wherein the CIN antagonist is selected from: a small molecule; an immunotherapy; siRNA/CRISPR; a gene therapy; and a combination of two or more thereof.

83. The method according to claim 82, wherein the small molecule is selected from: maraviroc; vicriviroc; and a combination thereof.

84. The method according to claim 74, wherein the CIN antagonist comprises a compound according to formula I, a hydrate thereof, a solvate thereof, pharmaceutically acceptable salts thereof, an isotopic isomer thereof, a prodrug thereof, or a complex thereof: 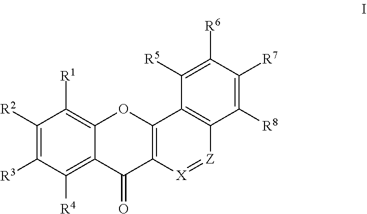
wherein,
R¹-R⁷ are independently selected from the group consisting of H, deuterium, halogen, OH, OR⁸, NO₂, CN, COOR⁸, NH₂, NR¹⁰, substituted or unsubstituted C₁₋₆ alkyl, C₁₋₆ haloalkyl, substituted or unsubstituted C₃₋₇ branched alkyl, and substituted or unsubstituted C₃₋₇ cycloalkyl, and substituted or unsubstituted aromatic ring with 0-3 heteroatoms;
R⁸ is selected from H, deuterium, C₁₋₆ alkyl, C₁₋₆ haloalkyl, C₃₋₇ branched alkyl, and C₃₋₇ cycloalkyl;
X and Z are independently selected from CH, CH₂, CO, O, CH, N, NO, and NR⁹, or together form a substituted or unsubstituted phenyl group;
R⁹ is selected from H, C₁₋₆ alkyl, C₁₋₆ haloalkyl, C₃₋₇ branched alkyl, and C₃₋₇ cycloalkyl, aromatic ring with 0-3 heteroatoms; and
R¹⁰ is selected from substituted or unsubstituted C₁₋₆ alkyl, C₁₋₆ haloalkyl, substituted or unsubstituted C₃₋₇ branched alkyl, and substituted or unsubstituted C₃₋₇ cycloalkyl, substituted or unsubstituted aromatic ring with 0-3 heteroatoms, and sulfonyl.

85. The method according to claim 84, wherein the compound of formula I is selected from a compound of formula Ik-Im, Io, Ip, a hydrate thereof, a solvate thereof, pharmaceutically acceptable salts thereof, an isotopic isomer thereof, a prodrug thereof, a complex thereof, and a combination of two or more thereof:

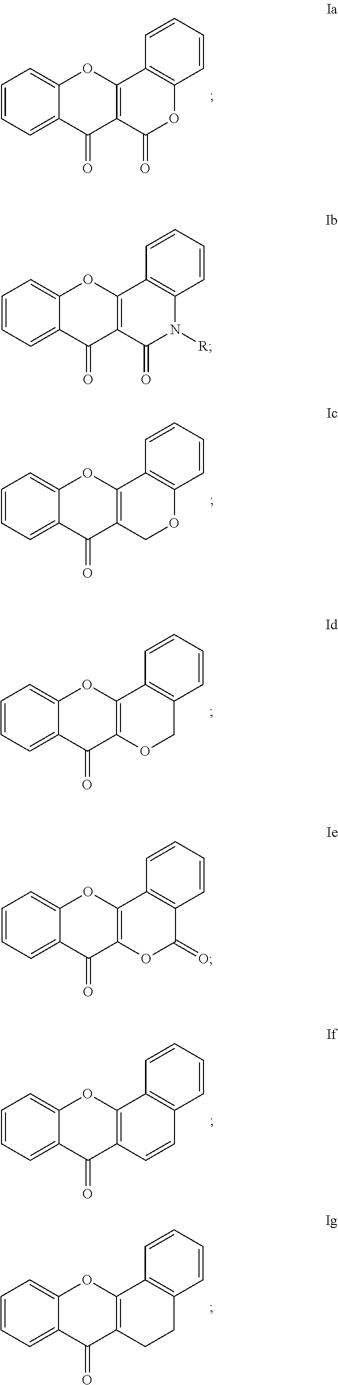

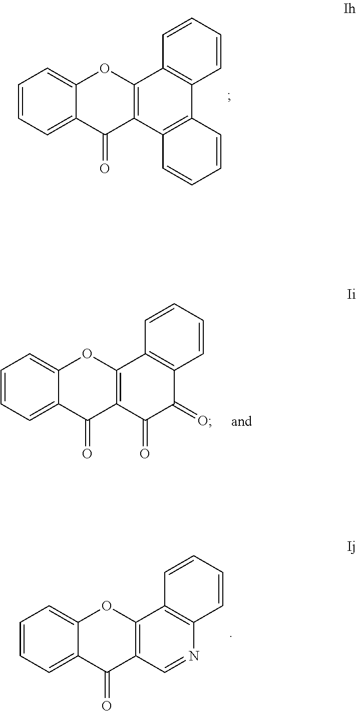 [SMILES: O=c1c2ccccc2oc2c3ccccc3c3ccccc3c12 ; O=C1C(=O)c2c(oc3ccccc3c2=O)-c2ccccc21 ; O=c1c2ccccc2oc2c1cnc1ccccc12]

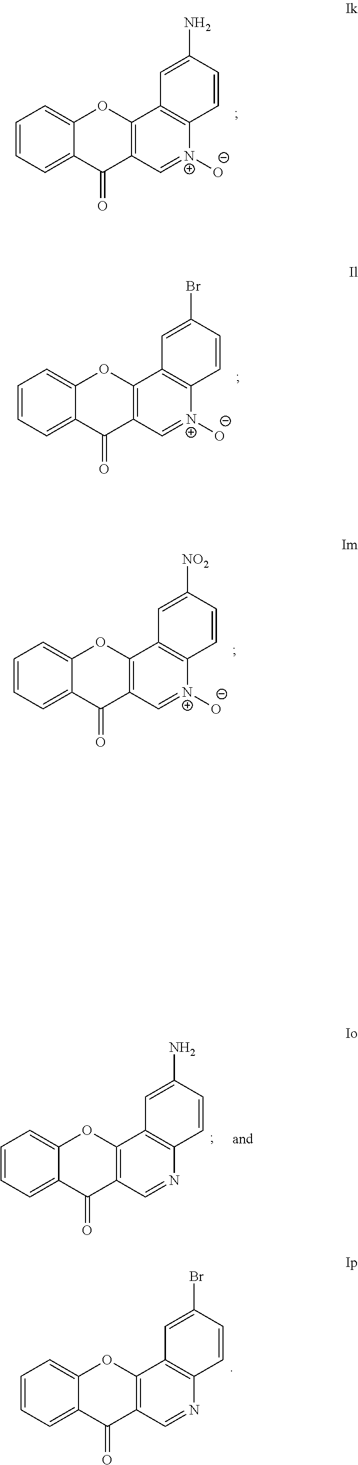 [SMILES: Nc1ccc2c(c1)c1oc3ccccc3c(=O)c1c[n+]2[O-] ; O=c1c2ccccc2oc2c1c[n+]([O-])c1ccc(Br)cc21 ; O=c1c2ccccc2oc2c1c[n+]([O-])c1ccc([N+](=O)[O-])cc21 ; Nc1ccc2ncc3c(=O)c4ccccc4oc3c2c1 ; O=c1c2ccccc2oc2c1cnc1ccc(Br)cc12]

86. A compound for reducing chromosomal instability, the compound having a structure according to formula I, a hydrate thereof, a solvate thereof, pharmaceutically acceptable salts thereof, an isotopic isomer thereof, a prodrug thereof, or a complex thereof: 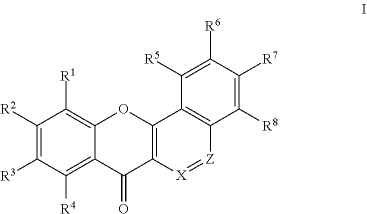
wherein,
R¹-R⁵ and R⁷ are independently selected from the group consisting of H, deuterium, halogen, OH, OR⁸, NO₂, CN, COOR⁸, NH₂, NR¹⁰, substituted or unsubstituted C₁₋₆ alkyl, C₁₋₆ haloalkyl, substituted or unsubstituted C₃₋₇ branched alkyl, and substituted or unsubstituted C₃₋₇ cycloalkyl, and substituted or unsubstituted aromatic ring with 0-3 heteroatoms;
R⁶ is selected from the group consisting of H, deuterium, halogen, OH, OR⁸, CN, COOR⁸, NH₂, NR¹⁰, substituted or unsubstituted C₁₋₆ alkyl, C₁₋₆ haloalkyl, substituted or unsubstituted C₃₋₇ branched alkyl, and substituted or unsubstituted C₃₋₇ cycloalkyl, and substituted or unsubstituted aromatic ring with 0-3 heteroatoms;
R⁸ is selected from H, deuterium, C₁₋₆ alkyl, C₁₋₆ haloalkyl, C₃₋₇ branched alkyl, and C₃₋₇ cycloalkyl;
X and Z are independently selected from CH, CH₂, CO, O, CH, N, NO, and NR⁹, or together form a substituted or unsubstituted phenyl group;
R⁹ is selected from H, C₁₋₆ alkyl, C₁₋₆ haloalkyl, C₃₋₇ branched alkyl, and C₃₋₇ cycloalkyl, aromatic ring with 0-3 heteroatoms; and
R¹⁰ is selected from substituted or unsubstituted C₁₋₆ alkyl, C₁₋₆ haloalkyl, substituted or unsubstituted C₃₋₇ branched alkyl, and substituted or unsubstituted C₃₋₇ cycloalkyl, substituted or unsubstituted aromatic ring with 0-3 heteroatoms, and sulfonyl.

87. The compound according to claim 86, wherein each of groups R¹-R⁵ and R⁷ are H.

88. The compound according to claim 86, wherein each of R¹-R⁷ are H.

89. The compound according to claim 86, wherein group R⁶ is selected from H, D, NH₂, and NR 10.

90. The compound according to claim 86, wherein one of group X or group Z is selected from O, N, and CO and the other of group X or group Z is selected from O, CO, CH, or CH₂.

91. The compound according to claim 86, wherein the compound of formula I is selected from a compound of formula Ik-Im, Io, Ip, a hydrate thereof, a solvate thereof, pharmaceutically acceptable salts thereof, an isotopic isomer thereof, a prodrug thereof, a complex thereof, and a combination of two or more thereof:

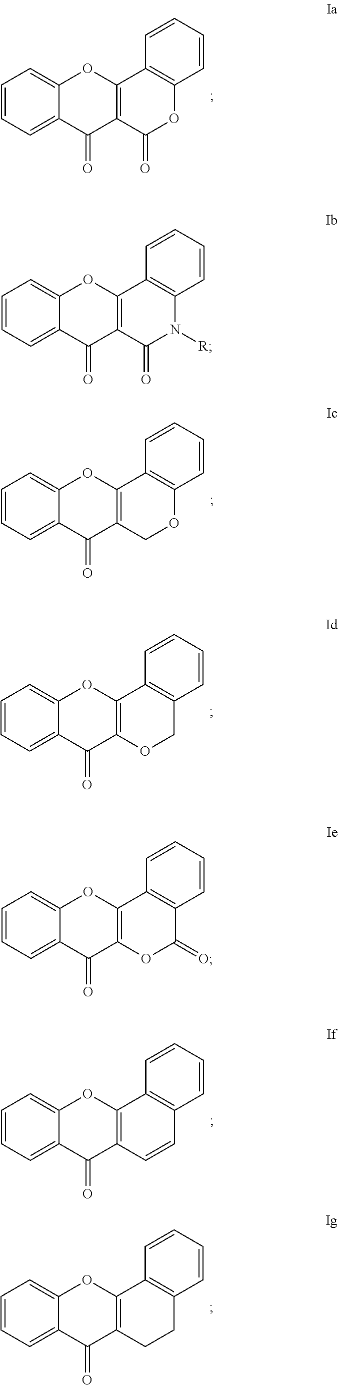

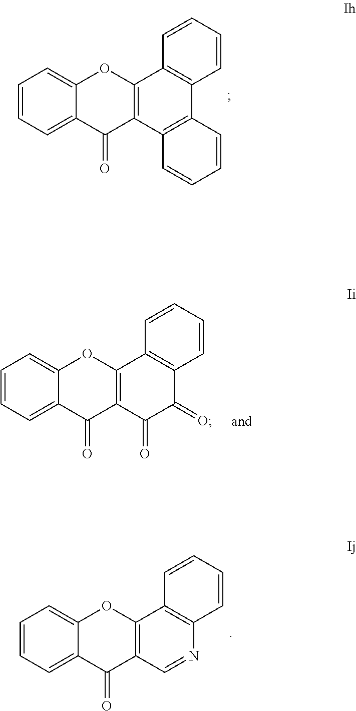 [SMILES: O=c1c2ccccc2oc2c3ccccc3c3ccccc3c12 ; O=C1C(=O)c2c(oc3ccccc3c2=O)-c2ccccc21 ; O=c1c2ccccc2oc2c1cnc1ccccc12]

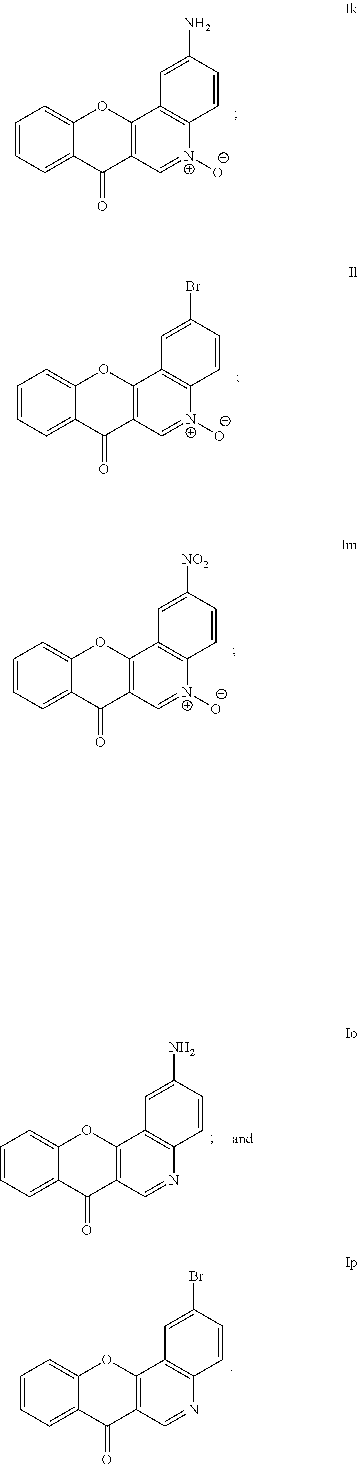 [SMILES: Nc1ccc2c(c1)c1oc3ccccc3c(=O)c1c[n+]2[O-] ; O=c1c2ccccc2oc2c1c[n+]([O-])c1ccc(Br)cc21 ; O=c1c2ccccc2oc2c1c[n+]([O-])c1ccc([N+](=O)[O-])cc21 ; Nc1ccc2ncc3c(=O)c4ccccc4oc3c2c1 ; O=c1c2ccccc2oc2c1cnc1ccc(Br)cc12]

92. The method according to claim 74, wherein the effective amount of the CIN antagonist is from about 1 mg/kg/day to about 200 mg/kg/day, optionally from about 10 mg/kg/day to about 190 mg/kg/day, or about 20 mg/kg/day to about 180 mg/kg/day, or about 30 mg/kg/day to about 170 mg/kg/day, or about 40 mg/kg/day to about 160 mg/kg/day, or about 50 mg/kg/day to about 150 mg/kg/day, or about 60 mg/kg/day to about 140 mg/kg/day, or about 70 mg/kg/day to about 130 mg/kg/day, or about 80 mg/kg/day to about 120 mg/kg/day, or about 90 mg/kg/day to about 110 mg/kg/day, or about 100 mg/kg/day.

93. A method of treating, preventing, or ameliorating CIN resulting from the administration of a chemotherapeutic agent comprising: administering an effective amount of a CIN antagonist followed by administering an effective amount of a chemotherapeutic agent.
